(19) United States
(12) Patent Application Publication
(10) Pub. No.: US 2024/0392279 A1
(43) Pub. Date: Nov. 28, 2024

(54) MODIFIED PROTEIN DISULFIDE ISOMERASE AND USES THEREOF

(71) Applicant: The University of North Carolina at Chapel Hill, Chapel Hill, NC (US)

(72) Inventors: Baggio Evangelista, Chapel Hill, NC (US); Todd Cohen, Chapel Hill, NC (US)

(21) Appl. No.: 18/691,748

(22) PCT Filed: Sep. 16, 2022
(86) PCT No.: PCT/US2022/076529

§ 371 Date: Mar. 13, 2024

Related U.S. Application Data
(60) Provisional application No. 63/245,329, filed on Sep. 17, 2021.

(51) Int. Cl. C12N 9/90; A61P 3/00

(52) U.S. Cl. CPC C12N 9/90; A61P 3/00; C12Y 503/04001

(57) ABSTRACT

Disclosed herein are modified protein disulfide isomerases (PDIs) or functional fragments thereof wherein the modified PDI or functional fragment thereof comprises a deletion of an endoplasmic reticulum (ER) signal sequence from a wild-type PDI or functional fragments thereof, and methods of making and using the same. The modified PDI or functional fragment thereof may be used to treat diseases associated with protein aggregates, including neurodegenerative diseases, muscular diseases, injuries, and aging-related conditions.

### STATEMENT OF PRIORITY

This application claims the benefit of U.S. Provisional Application Ser. No. 63/245,329, filed Sep. 17, 2021, the entire contents of which are incorporated by reference herein.

### FIELD OF INVENTION

The invention relates to modified protein disulfide isomerase (PDI) or a functional fragment thereof wherein the PDI or functional fragment thereof comprises a deletion of an endoplasmic reticulum (ER) signal sequence and methods of making and using the same. The modified PDI or functional fragment thereof may be used to treat diseases associated with protein aggregates, including neurodegenerative diseases, muscular diseases, injuries, and aging-related conditions.

### BACKGROUND

Protein aggregation contributes to several neurodegenerative diseases. Particularly, the accumulation of disulfide-crosslinked, cytosolic aggregates of the TAR DNA Binding Protein (TDP-43) has been linked to the pathology of several diseases including amyotrophic lateral sclerosis (ALS), frontotemporal lobar dementia (FTLD), and inclusion body myositis.

TDP-43 is a protein encoded by the TARDBP gene in humans. TDP-43 participates in many steps of protein production, and it can bind both DNA and RNA. The normal function of TDP-43 includes transcriptional repression, pre-mRNA splicing, and translational regulation. However, mutations in TARDBP are associated with approximately 5% of ALS cases. Additionally, misfolded TDP-43 that migrates from the nucleus to the cytoplasm is a hallmark of ALS and is associated with more than 97% of ALS cases. Other diseases associated with TDP-43 pathology include Parkinson's disease, Alzheimer's disease, prion disease, chronic traumatic encephalopathy (CTE), multisystem proteinopathy (MSP), Guam Parkinson-dementia complex (G-PDC) and ALS (G-ALS), facial onset sensory and motor neuronopathy, primary lateral sclerosis (PLS), progressive muscular atrophy (PMA), and others.

PDI is an enzyme in the endoplasmic reticulum (ER) that catalyzes disulfide bond formation and rearrangement between cysteine residues and proteins during protein folding. The PDI family consists of 15 gene family members that display both oxidoreductase and chaperone properties, with a varying degree of chaperone and/or isomerase activity. Missense mutations in genes encoding PDI have been identified in patients with ALS, although the mechanism by which PDI is associated with ALS is unknown. Thus, effective therapeutic strategies using PDI to combat neurodegenerative diseases associated with protein aggregates have not previously been obtained.

There is a need in the art for methods of reversing and preventing TDP-43 aggregates, methods of returning TDP-43 to a functional state, and method of treating disorders associated with TDP-43 aggregates.

### INVENTION SUMMARY

The present invention is based, in part, on the development of modified PDT or a functional fragment thereof that comprises a deletion of an ER signal sequence and the ability of the modified enzymes to reverse and/or prevent TDP-43 aggregation and pathologies associated therewith.

Accordingly, one aspect of the invention relates to a modified PDI or a functional fragment thereof wherein the modified PDI or functional fragment thereof comprises a deletion of an ER signal sequence from a wild-type PDI or functional fragment thereof.

Another aspect of the invention relates to a nucleic acid molecule encoding any one of the modified PDIs or functional fragments thereof of the invention.

An additional aspect of the invention relates a vector comprising any one of the nucleic acid molecules of the invention.

A further aspect of the invention relates to a host cell comprising any one of the modified PDIs or functional fragments thereof, nucleic acid molecules, or vectors of the invention.

Another aspect of the invention relates to a composition comprising any one of the modified PDIs or functional fragment thereof, nucleic acid molecules, or vectors of the invention.

A further aspect of the invention relates to a pharmaceutical composition comprising any one of the modified PDIs or functional fragment thereof, nucleic acid molecules, or vectors of the invention, and a pharmaceutically acceptable carrier.

An additional aspect of the invention relates to a method of treating a disease associated with TDP-43 aggregation in a subject in need thereof, the method comprising administering to the subject a therapeutically effective amount of any one of the modified PDIs or functional fragments thereof, nucleic acid molecules, or vectors of the invention, thereby treating the disease.

Another aspect of the invention relates to a method of treating a disease associated with mutations in the TARDBP gene in a subject in need thereof, the method comprising administering to the subject a therapeutically effective amount of any one of the modified PDIs or functional fragments thereof, nucleic acid molecules, or vectors of the invention, thereby treating the disease.

A further aspect of the invention relates to a method of delivering any one of the modified PDIs or functional fragments thereof of the invention, the method comprising administering to the subject any one of the modified PDIs or functional fragments thereof, nucleic acid molecules, or vectors of the invention, thereby delivering the modified PDI or functional fragment thereof to the subject.

An additional aspect of the invention relates to a method of disaggregating protein inclusions comprising TDP-43 in a subject, the method comprising administering to the subject any one of the modified PDIs or functional fragments thereof, nucleic acid molecules, or vectors of the invention, thereby disaggregating protein inclusions comprising TDP-43 in the subject.

Another aspect of the invention relates to a method of restoring function of TDP-43 in a subject, the method comprising administering to the subject any one of the modified PDIs or functional fragments thereof, nucleic acid molecules, or vectors of the invention, thereby inhibiting formation of protein inclusions comprising TDP-43 in the subject.

A further aspect of the invention relates to a method of inhibiting formation of protein inclusions comprising TDP-43 in a subject, the method comprising administering to the subject any one of the modified PDIs or functional fragments thereof, nucleic acid molecules, or vectors of the invention, thereby restoring function of TDP-43 in the subject.

### BRIEF DESCRIPTION OF THE DRAWINGS

FIG. 1. Images of HEK293T cells that were transfected with either native P4HB or modified P4HB, plus a green fluorescent protein (GFP)-tagged Sec61 fusion protein to serve as an ER counterstain. Cells were fixed, probed for P4HB, counterstained with the nuclear stain 4′,6-diamidino-2-phenylindole (DAPI) and imaged using a Zeiss 8000 laser scanning confocal microscope under 63× oil immersion objective.

FIGS. 2A-2B. A: Using a multi-well assay, HEK293A cells were transfected with a TDP-43-dNLS-2KQ variant and each PDI candidate or control as indicated. Cells were lysed in a chaotropic buffer, and protein lysates were adsorbed onto a nitrocellulose membrane in biological sextuplets. The membrane was probed with an antibody specific for phosphorylated TDP-43 at serine 409/410; B: Phosphorylated TDP-43 signal was normalized relative to total TDP-43 input.

FIGS. 3A-3B. A: HEK293 cells were transfected with TDP-43-dNLS-2KQ and a PDI candidate from the group of P4HB, AGR2, TXNDC12, CASQ1, CASQ2, ERp27, ERp29, and PDIA2, each with the N-terminal ER signal sequence removed and linked to FLAG or control vector expressing 3×FLAG-hamagglutinin. Cells were lysed in radioimmunoprecipitation assay buffer, sonicated, and centrifuged at 20,000 RCF (relative centrifugal field). The insoluble TDP-43-containing pellet was resuspended in 8M urea followed by sonication and centrifugation at 20,000 RCF. Lysates were prepared for reducing sodium dodecyl sulphate-polyacrylamide gel electrophoresis (SDS-PAGE) followed by immunoblotting for: phosphorylated TDP-43 (pS409/410), total TDP-43, FLAG, and glyceraldehyde 3-phosphate dehydrogenase (GAPDH); B: The insolubility levels of TDP-43 by PDI family member were analyzed with densitometry. The parameters for the densitometry measurements were as follows: 50 kDa band, equal sampling area across all conditions with background subtraction, normalized to GAPDH. Relative reduction in insoluble pS409/410 and total TDP-43 were plotted as mean +/−standard deviation. Alpha=0.05, calculated using Two-way ANOVA with Tukey's multiple comparisons test.

FIGS. 4A-4C. A: HEK293 cells were transfected with TDP-43-dNLS-2KQ and 3×FLAG-HA control or modified P4HB with the N-terminal ER signal sequence removed and linked to FLAG. Cells were fixed, probed for TDP-43 and P4HB, and imaged using laser scanning confocal microscopy. B: HEK293 cells were transfected with TDP-43-dNLS-2KQ and control or P4HB as above. TDP-43 was immunoprecipitated and subject to immunoblotting for P4HB; C: HEK293 cells were transfected with TDP-43-dNLS-2KQ and control vector as above, modified P4HB as above, or modified P4HB containing ablated thioredoxin motifs (C53/56/397/400S; called 4CS). Cells were subject to solubility fractionation as above and probed for pS409/410.

FIG. 5. Non-reducing PAGE of cells co-expressing TDP-43-dNLS-2KQ and functional or thioredoxin-null engineered P4HB as above were compared.

FIGS. 6A-6C. A: HEK293 cells were transfected with a nuclear-localized, aggregation prone TDP-43 variant (TDP-43-2KQ) which resulted in the formation of phase separated liquid condensates in the nuclear compartment. Images represent the mean area of TDP foci per condition. Images were acquired from 50 cells per condition on a Zeiss 8000 Laser Scanning Confocal Microscope and analyzed using ImageJ; B: The average size of nuclear TDP-43 foci in the control and the presence of P4HB; C: Biochemical fractionation of cell extracts.

FIG. 7. HEK293 cells were transfected as indicated. Cells were lysed and subject to co-immunoprecipitation using anti-nucleoporin 153 (anti-Nup153) capture beads. Precipitants were analyzed by western blot probing for TDP-43 and modified P4HB.

FIGS. 8A-8C. A: A nuclear-cytoplasmic shuttling sensor that consists of a dTomato fluorochrome appended to a nuclear localization- and nuclear export-sequence was used. B: TDP-43-dNLS-2KQ was co-expressed with either control (upper panel) or functional (ER signal sequence ablated/thioredoxin motif intact) P4HB (lower panel) in the presence of the shuttling sensor. C: The ratio of nuclear:cytoplasmic fluorescence intensity was calculated to determine nuclear shuttling efficacy. Violin plots represent 30 cells per condition imaged using a Zeiss 8000 laser scanning confocal microscope, analyzed in ImageJ.

FIGS. 9A-9B. A: Changes in levels of mature TDP-43 transcript were assessed by quantitative reverse transcription PCR (RT-qPCR) in cells expressing TDP-43-dNLS-2KQ and either functional (ER signal sequence ablated/thioredoxin motif intact) or thioredoxin-null P4HB; B: To validate P4HB-mediated rescue of transcriptional regulation, a minigene splicing reporter assay was employed. Cells were transfected with TDP-43-dNLS-2KQ and control or functional P4HB as above, in the presence of a cystic fibrosis transmembrane conductance regulator (CFTR) Exon 9 bait transcript.

FIGS. 10A-10B. A: GFP-TDP-43-dNLS-KQ was expressed with thioredoxin-null (ER signal sequence ablated) P4HB or functional (ER signal sequence ablated/thioredoxin motif intact) P4HB in adult murine tibialis anterior (TA) muscle under skeletal muscle-specific desmin promoter. TA muscle was removed, sectioned, and immunolabeled for: A TDP-43; B: The same set of tissue sections as in A were probed with an antibody against the TDP pathology hallmark pS409/410. Mice were anesthetized and to each leg TA muscle were injected with a cocktail of plasmid containing either: (TDP+liberated 4CS thioredoxin null) or (TDP+liberated thioredoxin+P4HB). Plasmids were governed by skeletal muscle specific desmin promoter. Following injection, each TA muscle was electroporated to deliver the plasmid DNA into myofibrils. Two weeks post-op, mice were sacrificed, TA harvested, sectioned, and prepared for histological analysis.

FIGS. 11A-11D. A: Depiction of the XBP1 UPR pathway. 11B-11D. Western Blots of XBP1 alternative splice variants, with XPBP1 splicing (XBPs) precursor for transcriptional activation and upregulation of UPR/ERAD regulators such as BiP for HEK293 cells transfected with: B: TDP-43-dNLS-2KQ with empty vector with FLAG or with 1.0 μM of tunicamycin added to transfected cells; C: ER-localized thioredoxin null P4HB and liberated thioredoxin null P4HB; D: ER-localized (wt) P4HB; and liberated P4HB.

FIGS. 12A-12B. ER-P4HB variants upregulate XBP1 target, UPR chaperone BiP. A: Western blot against main ER stress sensor and activator, BiP from protein lysate harvested from transfection of HEK293 cells with TDP-43-dNLS-2KQ with empty vector, ER-localized (wt)P4HB, ER-localized thioredoxin null P4HB, liberated P4HB, and liberated thioredoxin null P4HB. B: Chart of normalized BiP for transfection in 12A of empty vector control (FHA), ER-P4HB, ER-4CS, P4HB and 4CS.

FIGS. 13A-13B. Nuclear TDP-43 pathology observed in subset of ALS cases. A: Immunohistochemistry of ALS-FTLD patient and age-matched control illustrating the presence of nuclear-localized TDP-43 pathology (pTDP-43) and anti-localized, ER-resident P4HB. ER-localization prevents nuclear entry of P4HB to site of nuclear TDP-43 pathology. B: Immunocytochemistry illustrating that by liberating P4HB through mutation of ER-signal sequence, P4HB accesses the nucleus in addition to the cytoplasm (asterisk). Sec61 is used as an ER and nuclear lamin counterstain. Images represent a single confocal z plane.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying, and detecting nucleic acids, and the like. Such techniques are known to those skilled in the art. See, e.g., Sambrook et al, Molecular Cloning: A Laboratory Manual 4th Ed. (Cold Spring Harbor, NY, 2012); Ausubel e al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., chaperone and/or isomerase activity) of at least about 50% or more as compared to the activity of a polypeptide consisting of the recited sequence.

The term “protein disulfide isomerase” is known to those skilled in the art and refers to an enzyme located in the ER that catalyzes disulfide bond formation and rearrangement between cysteine residues and proteins during protein folding. Human genes that encode PDI include the gene family members AGR2, AGR3, TXNDC12, CASQ1, CASQ2, DNAJC10, ERP27, ERP29, ERP44, P41HB1, PDIA2, PDIA3, PDIA4, PDIA5, PDIA6, PDILT, TMX1, TMX2, TMX3, TMX4, and TXNDC5. Amino acid sequences of the PDIs and the location of the N-terminal ER signal sequence are shown in Table 1. In some embodiments, the PDI is not P4HB.

TABLE 1

| | | | N-terminal |
|---|---|---|---|
| | | SEQ | ER signal |
| | | ID | sequence |
| Name | Amino acid sequence | NO: | location |
| AGR2 | MEKIPVSAFLLLVALSYTLARDTTVKPGAKKDTKDSRPKLPQTLSRGWGDQLIWTQTY | 1 | AA1-20 |
| | EEALYKSKTSNKPLMIIHHLDECPHSQALKKVFAENKEIQKLAEQFVLLNLVYETTDKH | | |
| | LSPDGQYVPRIMFVDPSLTVRADITGRYSNRLYAYEPADTALLLDNMKKALKLLKTEL | | |
| AGR3 | MMLHSALGLCLLLVTVSSNLAIAIKKEKRPPQTLSRGWGDDITWVQTYEEGLFYAQK | 2 | AA1-21 |
| | SKKPLMVIHHLEDCQYSQALKKVFAQNEEIQEMAQNKFIMLNLMHETTDKNLSPDG | | |
| | QYVPRIMFVDPSLTVRADIAGRYSNRLYTYEPRDLPLLIENMKKALRLIQSEL | | |
| TXNDC12 | METRPRLGATCLLGFSFLLLVISSDGHNGLGKGFGDHIHWRTLEDGKKEAAASGLPL | 3 | AA1-26 |
| | MVIIHKSWCGACKALKPKFAESTEISELSHNFVMVNLEDEEEPKDEDFSPDGGYIPRIL | | |
| | FLDPSGKVHPEIINENGNPSYKYFYVSAEQVVQGMKEAQERLTGDAFRKKHLEDEL | | |
| CASQ1 | MSATDRMGPRAVPGLRLALLLLLVLGTPKSGVQGQEGLDFPEYDGVDRVINVNAKN | 4 | AA1-34 |
| | YKNVFKKYEVLALLYHEPPEDDKASQRQFEMEELILELAAQVLEDKGVGFGLVDSEKD | | |
| | AAVAKKLGLTEVDSMYVFKGDEVIEYDGEFSADTIVEFLLDVLEDPVELIEGERELQAF | | |
| | ENIEDEIKLIGYFKSKDSEHYKAFEDAAEEFHPYIPFFATFDSKVAKKLTLKLNEIDFYEAF | | |
| | MEEPVTIPDKPNSEEEIVNFVEEHRRSTLRKLKPESMYETWEDDMDGIHIVAFAEEAD | | |
| | PDGFEFLETLKAVAQDNTENPDLSIIWIDPDDFPLLVPYWEKTFDIDLSAPQIGVVNVT | | |
| | DADSVWMEMDDEEDLPSAEELEDWLEDVLEGEINTEDDDDDDDD | | |
| CASQ2 | MKRTHLFIVGIYFLSSCRAEEGLNFPTYDGKDRVVSLSEKNFKQVLKKYDLLCLYYHEP | 5 | AA1-19 |
| | VSSDKVTQKQFQLKEIVLELVAQVLEHKAIGFVMVDAKKEAKLAKKLGFDEEGSLYILK | | |
| | GDRTIEFDGEFAADVLVEFLLDLIEDPVEIISSKLEVQAFERIEDYIKLIGFFKSEDSEYYKA | | |
| | FEEAAEHFQPYIKFFATFDKGVAKKLSLKMNEVDFYEPFMDEPIAIPNKPYTEEELVEF | | |
| | VKEHQRPTLRRLRPEEMFETWEDDINGIHIVAFAEKSDPDGYEFLEILKQVARDNTD | | |
| | NPDLSILWIDPDDFPLLVAYWEKTFKIDLFRPQIGVVNVTDADSVWMEIPDDDDLPT | | |
| | AEELEDWIEDVLSGKINTEDDDEDDDDDDNSDEEDNDDSDDDDDE | | |
| DNAJC10 | MGVWLNKDDYIRDLKRIILCFLIVYMAILVGTDQDFYSLLGVSKTASSREIRQAFKKLAL | 6 | AA1-32 |
| | KLHPDKNPNNPNAHGDFLKINRAYEVLKDEDLRKKYDKYGEKGLEDNQGGQYESW | | |
| | NYYRYDFGIYDDDPEIITLERREFDAAVNSGELWFVNFYSPGCSHCHDLAPTWRDFA | | |
| | KEVDGLLRIGAVNCGDDRMLCRMKGVNSYPSLFIFRSGMAPVKYHGDRSKESLVSFA | | |
| | MQHVRSTVTELWTGNFVNSIQTAFAAGIGWLITFCSKGGDCLTSQTRLRLSGMLFLN | | |
| | SLDAKEIYLEVIHNLPDFELLSANTLEDRLAHHRWLLFFHFGKNENSNDPELKKLKTLLK | | |
| | NDHIQVGRFDCSSAPDICSNLYVFQPSLAVFKGQGTKEYEIHHGKKILYDILAFAKESV | | |
| | NSHVTTLGPQNFPANDKEPWLVDFFAPWCPPCRALLPELRRASNLLYGQLKFGTLDC | | |
| | TVHEGLCNMYNIQAYPTTVVFNQSNIHEYEGHHSAEQILEFIEDLMNPSVVSLTPTTF | | |
| | NELVTQRKHNEVWMVDFYSPWCHPCQVLMPEWKRMARTLTGLINVGSIDCQQYH | | |
| | SFCAQENVQRYPEIRFFPPKSNKAYHYHSYNGWNRDAYSLRIWGLGFLPQVSTDLTP | | |
| | QTFSEKVLQGKNHWVIDFYAPWCGPCQNFAPEFELLARMIKGKVKAGKVDCQAYA | | |
| | QTCQKAGIRAYPTVKFYFYERAKRNFQEEQINTRDAKAIAALISEKLETLRNQGKRNK | | |
| | DEL | | |
| ERP27 | MEAAPSRFMFLLFLLTCELAAEVAAEVEKSSDGPGAAQEPTWLTDVPAAMEFIAATE | 7 | AA1-25 |
| | VAVIGFFQDLEIPAVPILHSMVQKFPGVSFGISTDSEVLTHYNITGNTICLFRLVDNEQL | | |
| | NLEDEDIESIDATKLSRFIEINSLHMVTEYNPVTVIGLFNSVIQIHLLLIMNKASPEYEEN | | |
| | MHRYQKAAKLFQGKILFILVDSGMKENGKVISFFKLKESQLPALAIYQTLDDEWDTLP | | |
| | TAEVSVEHVQNFCDGFLSGKLLKENRESEGKTPKVEL | | |
| ERP29 | MAAAVPRAAFLSPLLPLLLGFLLLSAPHGGSGLHTKGALPLDTVTFYKVIPKSKFVLVKF | 8 | AA1-32 |
| | DTQYPYGEKQDEFKRLAENSASSDDLLVAEVGISDYGDKLNMELSEKYKLDKESYPVF | | |
| | YLFRDGDFENPVPYTGAVKVGAIQRWLKGQGVYLGMPGCLPVYDALAGEFIRASGV | | |
| | EARQALLKQGQDNLSSVKETQKKWAEQYLKIMGKILDQGEDFPASEMTRIARLIEKN | | |
| | KMSDGKKEELQKSLNILTAFQKKGAEKEEL | | |
| ERP44 | MHPAVFLSLPDLRCSLLLLVTWVFTPVTTEITSLDTENIDEILNNADVALVNFYADWC | 9 | AA1-29 |
| | RFSQMLHPIFEEASDVIKEEFPNENQVVFARVDCDQHSDIAQRYRISKYPTLKLFRNG | | |
| | MMMKREYRGQRSVKALADYIRQQKSDPIQEIRDLAEITTLDRSKRNIIGYFEQKDSDN | | |
| | YRVFERVANILHDDCAFLSAFGDVSKPERYSGDNIIYKPPGHSAPDMVYLGAMTNFD | | |
| | VTYNWIQDKCVPLVREITFENGEELTEEGLPFLILFHMKEDTESLEIFQNEVARQLISEK | | |
| | GTINFLHADCDKFRHPLLHIQKTPADCPVIAIDSFRHMYVFGDFKDVLIPGKLKQFVFD | | |
| | LHSGKLHREFHHGPDPTDTAPGEQAQDVASSPPESSFQKLAPSEYRYTLLRDRDEL | | |
| P4HB | MLRRALLCLAVAALVRADAPEEEDHVLVLRKSNFAEALAAHKYLLVEFYAPWCGHCK | 10 | AA1-17 |
| | ALAPEYAKAAGKLKAEGSEIRLAKVDATEESDLAQQYGVRGYPTIKFFRNGDTASPKE | | |
| | YTAGREADDIVNWLKKRTGPAATTLPDGAAAESLVESSEVAVIGFFKDVESDSAKQFL | | |
| | QAAEAIDDIPFGITSNSDVFSKYQLDKDGVVLFKKFDEGRNNFEGEVTKENLLDFIKH | | |
| | NQLPLVIEFTEQTAPKIFGGEIKTHILLFLPKSVSDYDGKLSNFKTAAESFKGKILFIFIDSD | | |
| | HTDNQRILEFFGLKKEECPAVRLITLEEEMTKYKPESEELTAERITEFCHRFLEGKIKPHL | | |
| | MSQELPEDWDKQPVKVLVGKNFEDVAFDEKKNVFVEFYAPWCGHCKQLAPIWDKL | | |
| | GETYKDHENIVIAKMDSTANEVEAVKVHSFPTLKFFPASADRTVIDYNGERTLDGFKK | | |
| | FLESGGQDGAGDDDDLEDLEEAEEPDMEEDDDQKAVKDEL | | |
| PDIA2 | MSRQLLPVLLLLLLRASCPWGQEQGARSPSEEPPEEEIPKEDGILVLSRHTLGLALREH | 11 | AA1-21 |
| | PALLVEFYAPWCGHCQALAPEYSKAAAVLAAESMVVTLAKVDGPAQRELAEEFGVT | | |
| | EYPTLKFFRNGNRTHPEEYTGPRDAEGIAEWLRRRVGPSAMRLEDEAAAQALIGGR | | |
| | DLVVIGFFQDLQDEDVATFLALAQDALDMTFGLTDRPRLFQQFGLTKDTVVLFKKFD | | |
| | EGRADFPVDEELGLDLGDLSRFLVTHSMRLVTEFNSQTSAKIFAARILNHLLLFVNQTL | | |
| | AAHRELLAGFGEAAPRFRGQVLFVVVDVAADNEHVLQYFGLKAEAAPTLRLVNLETT | | |
| | KKYAPVDGGPVTAASITAFCHAVLNGQVKPYLLSQEIPPDWDQRPVKTLVGKNFEQ | | |
| | VAFDETKNVFVKFYAPWCTHCKEMAPAWEALAEKYQDHEDIIIAELDATANELDAFA | | |
| | VHGFPTLKYFPAGPGRKVIEYKSTRDLETFSKFLQNGGVLPTEEPPEEPAAPFPEPPAN | | |
| | STMGSKEEL | | |
| PDIA3 | MRLRRLALFPGVALLLAAARLAAASDVLELTDDNFESRISDTGSAGLMLVEFFAPWC | 12 | AA1-24 |
| | GHCKRLAPEYEAAATRLKGIVPLAKVDCTANTNTCNKYGVSGYPTLKIFRDGEEAGAY | | |
| | DGPRTADGIVSHLKKQAGPASVPLRTEEEFKKFISDKDASIVGFFDDSFSEAHSEFLKA | | |
| | ASNLRDNYRFAHTNVESLVNEYDDNGEGIILFRPSHLTNKFEDKTVAYTEQKMTSGKI | | |
| | KKFIQENIFGICPHMTEDNKDLIQGKDLLIAYYDVDYEKNAKGSNYWRNRVMMVAK | | |
| | KFLDAGHKLNFAVASRKTFSHELSDFGLESTAGEIPVVAIRTAKGEKFVMQEEFSRDG | | |
| | KALERFLQDYFDGNLKRYLKSEPIPESNDGPVKVVVAENFDEIVNNENKDVLIEFYAP | | |
| | WCGHCKNLEPKYKELGEKLSKDPNIVIAKMDATANDVPSPYEVRGFPTIYFSPANKKL | | |
| | NPKKYEGGRELSDFISYLQREATNPPVIQEEKPKKKKKAQEDL | | |
| PDIA4 | MRPRKAFLLLLLLGLVQLLAVAGAEGPDEDSSNRENAIEDEEEEEEEDDDEEEDDLEV | 13 | AA1-20 |
| | KEENGVLVLNDANFDNFVADKDTVLLEFYAPWCGHCKQFAPEYEKIANILKDKDPPIP | | |
| | VAKIDATSASVLASRFDVSGYPTIKILKKGQAVDYEGSRTQEEIVAKVREVSQPDWTP | | |
| | PPEVTLVLTKENFDEVVNDADIILVEFYAPWCGHCKKLAPEYEKAAKELSKRSPPIPLA | | |
| | KVDATAETDLAKRFDVSGYPTLKIFRKGRPYDYNGPREKYGIVDYMIEQSGPPSKEILT | | |
| | LKQVQEFLKDGDDVIIIGVFKGESDPAYQQYQDAANNLREDYKFHHTESTEIAKFLKV | | |
| | SQGQLVVMQPEKFQSKYEPRSHMMDVQGSTQDSAIKDFVLKYALPLVGHRKVSND | | |
| | AKRYTRRPLVVVYYSVDFSFDYRAATQFWRSKVLEVAKDFPEYTFAIADEEDYAGEVK | | |
| | DLGLSESGEDVNAAILDESGKKFAMEPEEFDSDTLREFVTAFKKGKLKPVIKSQPVPKN | | |
| | NKGPVKVVVGKTFDSIVMDPKKDVLIEFYAPWCGHCKQLEPVYNSLAKKYKGQKGL | | |
| | VIAKMDATANDVPSDRYKVEGFPTIYFAPSGDKKNPVKFEGGDRDLEHLSKFIEEHAT | | |
| | KLSRTKEEL | | |
| PDIAS | MARAGPAWLLLAIWVVLPSWLSSAKVSSLIERISDPKDLKKLLRTRNNVLVLYSKSEVA | 14 | AA1-21 |
| | AENHLRLLSTVAQAVKGQGTICWVDCGDAESRKLCKKMKVDLSPKDKKVELFHYQD | | |
| | GAFHTEYNRAVTFKSIVAFLKDPKGPPLWEEDPGAKDVVHLDSEKDFRRLLKKEEKPL | | |
| | LIMFYAPWCSMCKRMMPHFQKAATQLRGHAVLAGMNVYSSEFENIKEEYSVRGFP | | |
| | TICYFEKGRFLFQYDNYGSTAEDIVEWLKNPQPPQPQVPETPWADEGGSVYHLTDE | | |
| | DFDQFVKEHSSVLVMFHAPWCGHCKKMKPEFEKAAEALHGEADSSGVLAAVDATV | | |
| | NKALAERFHISEFPTLKYFKNGEKYAVPVLRTKKKFLEWMQNPEAPPPPEPTWEEQQ | | |
| | TSVLHLVGDNFRETLKKKKHTLVMFYAPWCPHCKKVIPHFTATADAFKDDRKIACAA | | |
| | VDCVKDKNQDLCQQEAVKGYPTFHYYHYGKFAEKYDSDRTELGFTNYIRALREGDHE | | |
| | RLGKKKEEL | | |
| PDIA6 | MRRDLREKLVWVCRPLAPVEVPANISSDFQPCSPTSPAHSLSRKSPIMYPSTTMANA | 15 | AA1-19 |
| | PGLVSCTFFLAVNGLYSSSDDVIELTPSNFNREVIQSDSLWLVEFYAPWCGHCQRLTP | | |
| | EWKKAATALKDVVKVGAVDADKHHSLGGQYGVQGFPTIKIFGSNKNRPEDYQGGR | | |
| | TGEAIVDAALSALRQLVKDRLGGRSGGYSSGKQGRSDSSSKKDVIELTDDSFDKNVLD | | |
| | SEDVWMVEFYAPWCGHCKNLEPEWAAAASEVKEQTKGKVKLAAVDATVNQVLAS | | |
| | RYGIRGFPTIKIFQKGESPVDYDGGRTRSDIVSRALDLFSDNAPPPELLEIINEDIAKRTC | | |
| | EEHQLCVVAVLPHILDTGAAGRNSYLEVLLKLADKYKKKMWGWLWTEAGAQSELET | | |
| | ALGIGGFGYPAMAAINARKMKFALLKGSFSEQGINEFLRELSFGRGSTAPVGGGAFPT | | |
| | IVEREPWDGRDGELPVEDDIDLSDVELDDLGKDEL | | |
| PDILT | MDLLWMPLLLVAACVSAVHSSPEVNAGVSSIHITKPVHILEERSLLVLTPAGLTQMLN | 16 | AA1-20 |
| | QTRFLMVLFHNPSSKQSRNLAEELGKAVEIMGKGKNGIGFGKVDITIEKELQQEFGIT | | |
| | KAPELKLFFEGNRSEPISCKGVVESAALVVWLRRQISQKAFLFNSSEQVAEFVISRPLVI | | |
| | VGFFQDLEEEVAELFYDVIKDFPELTFGVITIGNVIGRFHVTLDSVLVFKKGKIVNRQKLI | | |
| | NDSTNKQELNRVIKQHLTDFVIEYNTENKDLISELHIMSHMLLFVSKSSESYGIIIQHYK | | |
| | LASKEFQNKILFILVDADEPRNGRVFKYFRVTEVDIPSVQILNLSSDARYKMPSDDITYE | | |
| | SLKKFGRSFLSKNATKHQSSEEIPKYWDQGLVKQLVGKNFNVVVFDKEKDVFVMFYA | | |
| | PWSKKCKMLFPLLEELGRKYQNHSTIIIAKIDVTANDIQLMYLDRYPFFRLFPSGSQQA | | |
| | VLYKGEHTLKGFSDFLESHIKTKIEDEDELLSVEQNEVIEEEVLAEEKEVPMMRKGLPE | | |
| | QQSPELENMTKYVSKLEEPAGKKKTSEEVVVVVAKPKGPPVQKKKPKVKEEL | | |
| TMX1 | MAPSGSLAVPLAVLVLLLWGAPWTHGRRSNVRVITDENWRELLEGDWMIEFYAPW | 17 | AA1-26 |
| | CPACQNLQPEWESFAEWGEDLEVNIAKVDVTEQPGLSGRFIITALPTIYHCKDGEFRR | | |
| | YQGPRTKKDFINFISDKEWKSIEPVSSWFGPGSVLMSSMSALFQLSMWIRTCHNYFI | | |
| | EDLGLPVWGSYTVFALATLFSGLLLGLCMIFVADCLCPSKRRRPQPYPYPSKKLLSESA | | |
| | QPLKKVEEEQEADEEDVSEEEAESKEGTNKDFPQNAIRQRSLGPSLATDKS | | |
| TMX2 | MAVLAPLIALVYSVPRLSRWLAQPYYLLSALLSAAFLLVRKLPPLCHGLPTQREDGNPC | 18 | AA1-48 |
| | DFDWREVEILMFLSAIVMMKNRRSMFLMTCKPPLYMGPEYIKYFNDKTIDEELERDK | | |
| | RVTWIVEFFANWSNDCQSFAPIYADLSLKYNCTGLNFGKVDVGRYTDVSTRYKVSTS | | |
| | PLTKQLPTLILFQGGKEAMRRPQIDKKGRAVSWTFSEENVIREFNLNELYQRAKKLSK | | |
| | AGDNIPEEQPVASTPTTVSDGENKKDK | | |
| TMX3 | MAAWKSWTALRLCATVVVLDMVVCKGFVEDLDESFKENRNDDIWLVDFYAPWCG | 19 | AA1-24 |
| | HCKKLEPIWNEVGLEMKSIGSPVKVGKMDATSYSSIASEFGVRGYPTIKLLKGDLAYN | | |
| | YRGPRTKDDIIEFAHRVSGALIRPLPSQQMFEHMQKRHRVFFVYVGGESPLKEKYIDA | | |
| | ASELIVYTYFFSASEEVVPEVIFKI | | |
| TMX4 | MAGGRCGPQLTALLAAWIAAVAATAGPEEAALPPEQSRVQPMTASNWTLVMEGE | 20 | AA1-23 |
| | WMLKFYAPWCPSCQQTDSEWEAFAKNGEILQISVGKVDVIQEPGLSGRFFVTTLPAF | | |
| | FHAKDGIFRRYRGPGIFEDLQNYILEKKWQSVEPLTGWKSPASLTMSGMAGLFSISG | | |
| | KIWHLHNYFTVTLGIPAWCSYVFFVIATLVFGLFMGLVLVVISECFYVPLPRHLSERSE | | |
| | QNRRSEEAHRAEQLQDAEEEKDDSNEEENKDSLVDDEEEKEDLGDEDEAEEEEEED | | |
| | NLAAGVDEERSEANDQGPPGEDGVTREEVEPEEAEEGISEQPCPADTEVVEDSLRQR | | |
| | KSQHADKGL | | |
| TXNDC5 | MEDAKVYVAKVDCTAHSDVCSAQGVRGYPTLKLFKPGQEAVKYQGPRDFQTLENW | 21 | AA1-32 |
| | MLQTLNEEPVTPEPEVEPPSAPELKQGLYELSASNFELHVAQGDHFIKFFAPWCGHC | | |
| | KALAPTWEQLALGLEHSETVKIGKVDCTQHYELCSGNQVRGYPTLLWFRDGKKVDQ | | |
| | YKGKRDLESLREYVESQLQRTETGATETVTPSEAPVLAAEPEADKGTVLALTENNFDD | | |
| | TIAEGITFIKFYAPWCGHCKTLAPTWEELSKKEFPGLAGVKIAEVDCTAERNICSKYSVR | | |
| | GYPTLLLFRGGKKVSEHSGGRDLDSLHRFVLSQAKDEL | | |

The term “modified protein disulfide isomerase (modified PDI)” as used herein refers to the addition, deletion, and/or substitution of one or more amino acids from the wild-type PDI enzyme, wherein the modified PDI substantially retains at least one biological activity normally associated with that polypeptide (e.g., wild-type PDI protein or fragment thereof). In some embodiments, modified PDI is a modified AGR2, AGR3, TXNDC12, CASQ1, CASQ2, DNAJC10, ERP27, ERP29, ERP44, P4HB, PDIA2, PDIA3, PDIA4, PDIA5, PDIA6, PDILT, TMIX1, TMX2, TMX3, TMX4, or TXNDC5 gene family member.

As used herein, the term “polypeptide” encompasses both peptides and proteins (including PDT), unless indicated otherwise.

As used herein, a “functional” polypeptide or “functional fragment” is one that substantially retains at least one biological activity normally associated with that polypeptide (e.g., wild-type PDI protein or fragment thereof). In particular embodiments, the “functional” polypeptide or “functional fragment” substantially retains all of the activities possessed by the unmodified polypeptide (e.g., wild-type PDI protein or fragment thereof). By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as chaperone and/or isomerase activity can be measured using assays that are well known in the art and as described herein.

The term “fragment,” as applied to a peptide, will be understood to mean an amino acid sequence of reduced length relative to a reference peptide (e.g., wild-type PDT protein) or amino acid sequence and comprising, consisting essentially of, and/or consisting of an amino acid sequence of contiguous amino acids identical to the reference peptide or amino acid sequence. Such a peptide fragment according to the invention may be, where appropriate, included in a larger polypeptide of which it is a constituent. In some embodiments, such fragments can comprise, consist essentially of, and/or consist of peptides having a length of at least about 5, 10, 15, 20, 25, 30, 35, 46. 50, 55, 60, 65, 70, 75, 80, 85, 90, 95 or 100 or more consecutive amino acids of a peptide or amino acid sequence according to the invention.

The terms “polynucleotide,” “nucleic acid,” “nucleic acid molecule,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example, those described in Current Protocols in Molecular Biology (Ausubel el al., eds. 1987) Supplement 30, section 7.7.18, Table 7.7.1. Preferably, default parameters are used for alignment. One alignment program is BLAST, using default parameters. Examples of the programs include BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

The term “endoplasmic reticulum signal sequence” (“ER signal sequence”) as used herein refers to any amino acid sequence that anchors a polypeptide in the ER.

The term “nuclear localization signal,” or “nuclear localization sequence,” (“NLS”) as used herein refers to an amino acid sequence that tags a protein for import into the cell nucleus by cell transport.

As used herein, the term “operably linked” means that the promoter and coding sequence are joined together in a manner that allows them to carry out their normal functions, i.e., transcription of the coding sequence is under the control of the promoter and the transcript produced is correctly translated into the desired product.

Those skilled in the art will appreciate that a variety of promoters may be used depending on the level and specific expression desired. The promoter may be constitutive or regulatable, depending on the pattern of expression desired. The promoter may be native or foreign and can be a natural or a synthetic sequence. By foreign, it is intended that the transcriptional initiation region is not found in the wild-type host into which the transcriptional initiation region is introduced.

The promoter can be native to the target cell or subject to be treated and/or can be native to the heterologous nucleotide sequence. The promoter is generally chosen so that it will function in the target cell(s) of interest. The promoter can optionally be a mammalian promoter. The promoter may further be constitutive or regulatable (e.g., inducible).

Promoters for nucleic acid delivery can be tissue preferred and/or -specific promoters. In some embodiments, the promoter is brain-specific or brain-preferred, spinal cord-specific or spinal cord-preferred, or muscle-specific or muscle-preferred.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, and viruses (bacteriophage, animal viruses, and plant viruses). In some embodiments the vector is a viral vector, optionally an adeno-associated virus (AAV) vector. Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and/or adenovirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.). Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

As used herein, the term “adeno-associated virus” (AAV) includes but is not limited to, AAV serotype 1 (AAV1), AAV2, AAV3 (including types 3A and 3B), AAV4, AAV5, AAV6, AAV7, AAV8, AAV9, AAV10, AAV11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). Recently, a number of putative new AAV serotypes and clades have been identified (see, e.g., Gao et al., (2004) J. Virology 78:6381-6388; Moris et al., (2004) Virology 33-:375-383; and Table 1).

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

As used herein, the term “host cell” refers to a cell that is engineered to express the modified PDI polypeptide or functional fragment thereof (e.g., a modified full length PDI protein or a fragment thereof). “Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Host cells may be derived from prokaryotes or eukaryotes, depending upon whether the desired result is replication of the vector or expression of part or all of the vector-encoded nucleic acid sequences. Prokaryotes include gram negative or positive cells. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5Rα, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

“Pharmaceutically acceptable carrier” (sometimes referred to as a “carrier”) refers to a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90°/a, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof as used with respect to a polypeptide and a cell or aggregate, refers to bringing the polypeptide and the cell or aggregate in sufficiently close proximity to each other for one to exert a biological effect on the other. In some embodiments, the term contact means binding of the polypeptide to the cell or aggregate.

“Misfolding” refers to a failure of a protein to attain or maintain its normal three-dimensional conformation, e.g., the tertiary structure in which it performs its normal biological function in a cell or organism. In some embodiments, misfolding comprises formation of soluble oligomers comprising two, three, or more molecules of the protein (e.g., up to about 10-20 molecules of the protein), wherein the oligomers do not perform a normal function of the protein (e.g., oligomerization is not part of the normal assembly or activation pathway of the protein). In some embodiments misfolding comprises formation of larger protein aggregates, e.g., aggregates of a size sufficient to permit their detection using optical microscopy. In some embodiments an aggregate comprises an amyloid. As known in the art, an amyloid is a protein aggregate characterized by a cross-beta sheet structure. Amyloids are typically composed of about 5-10 nm wide cross-beta fibrils (also called “filaments”), in which the polypeptide chain is arranged in beta-sheets where the polypeptide is perpendicular to the fibril axis and hydrogen bonding is parallel. Amyloids may be identified using methods such as fluorescent dyes, polarimetry, circular dichroism, FTIR, conformation-specific antibodies, or X-ray diffraction. For example, an amyloid can typically be identified by detecting a change in the fluorescence intensity of planar aromatic dyes such as thioflavin T or Congo red upon binding to the amyloid. A “misfolded protein” is a protein that has failed to fold properly, has become unfolded, has folded into an abnormal conformation, and/or has formed a dysfunctional oligomer or larger protein aggregate or is in a conformation in which it is prone to form a dysfunctional oligomer or larger protein aggregate, e.g., an amyloid.

As used herein, the term “disaggregate” refers to the breaking down of one or more protein aggregates. As a protein aggregate contains numerous copies of a protein clumped together, “disaggregation” refers to a process of removing portions of the aggregated protein clump. Thus, as used herein, “disaggregation” refers to the removal of portions of an existing protein aggregate, such that after disaggregation, the result is a smaller protein aggregate clump or an absence of a protein aggregate clump altogether. Detection of aggregate size and changes thereto depend on the sensitivity of the equipment and techniques used to detect aggregate size. Thus, under one technique, a disaggregated clump may be undetectable, whereas under another technique, the same disaggregated clump may be detected as having a smaller size.

“Restoring function,” as used herein, refers to functional restoration of TDP-43 in broad DNA/RNA metabolic processes including protein/transcript nucleo-cytoplasmic shuttling, pre-mRNA alternative splicing of TDP-43 target genes, miRNA processing, lncRNA/nucleolar organization, TARDBP autoregulation, nuclear membrane/pore organization, translational modulation, and transport or stress granule assembly/disassembly. The restoration of function may be complete (e.g., 100% restoration of all function(s)) or partial (e.g., 50% or more restoration of at least one of the normal metabolic processes of TDP-43.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g., a human, non-human primate, lagomorph (e.g., rabbit), or rodent (e.g., mouse, rat). In some embodiments a human subject is a neonate, child, adult, or geriatric subject. In some embodiments a human subject is at least 50, 60, 70, 80, or 90 years old.

“Treat,” “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome, or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

A “disease associated with TDP-43 aggregation” refers to any disease or disorder that is caused by or has at least one symptom caused by aggregation of TDP-43 and/or mutations in the TARDBP gene. Examples include, but are not limited to, Parkinson's disease, Alzheimer's disease, prion disease, chronic traumatic encephalopathy (CTE), multisystem proteinopathy (MSP), Guam Parkinson-dementia complex (G-PDC) and ALS (G-ALS), facial onset sensory and motor neuronopathy, primary lateral sclerosis (PLS), progressive muscular atrophy (PMA), frontotemporal dementia, Limbic-predominant age-related TDP-43 encephalopathy (LATE), cerebral age-related TDP-43 with sclerosis (CARTS), Perry disease, and others. See, e.g., DeBoer, et al., J Neurol Neurosurg Psychiatry 2021; 92:86-95; doi: 10.1136/jnnp-2020-322953. A disease associated with TDP-43 aggregation may also be identified by assays as described herein and known in the art, including commercially available assays from, e.g., PerkinElmer® and Quanterix™.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

The present invention is based, in part, on the development of a modified PDI or a functional fragment thereof that comprises a deletion of an ER signal sequence. The deletion allows the modified PDT to be present in the cytoplasm and/or nucleus where it can act on aggregates.

Accordingly, one aspect of the invention relates to a modified PDI or a functional fragment thereof wherein the modified PDI or functional fragment thereof comprises a deletion of an ER signal sequence from a wild-type PDI or functional fragment thereof. In some embodiments, more than one ER signal sequence is deleted. The deletion may be of all the amino acid residues in the signal sequence or a sufficient number of amino acid residues to render the signal sequence ineffective. In an example embodiment, the deletion of amino acid residues of the signal sequence comprises 2, 3, 4, 5, 6, 7, 8, 9, 10, 11, 12, 13, 14, 15 or more residues, or 5%, 10%, 15%, 20%, 30%, 40%, 50% or more of the residues of the signal sequence that are either continuous deletions or deletions at one or more locations within the signal sequence.

In some embodiments, the ER signal sequence is deleted from the N-terminus of the wild-type PDI or functional fragment thereof. In other embodiments, the ER signal sequence is deleted from the C-terminus of the wild-type PDI or functional fragment thereof. In certain embodiments, example C-terminus sequences of the wild-type PDI may comprise KDEL (SEQ ID NO: 22), RDEL (SEQ ID NO: 23), HDEL (SEQ ID NO: 24), KEEL (SEQ ID NO: 25), or KVEL (SEQ ID NO: 26).

In some embodiments, the deleted ER signal sequence from the N-terminus is replaced with an initiator methionine. In some embodiments, the initiator methionine is linked to an epitope tag, e.g., 1×/2×/3× FLAG, 1-6× histidine, hemagglutinin, glutathione-S-transferase, V5, or green fluorescent protein or other fluorescent protein variants.

In some embodiments the modified PDI or functional fragment thereof comprises a deletion of an ER signal sequence, optionally wherein the ER signal sequence is deleted from the N-terminus or C-terminus, and the protein further comprises one or more exogenous targeting signals, e.g., a nuclear localization signal (NLS) (e.g., the sequence KRKMDETDASSAVKVKR ((SEQ ID NO: 27)) or a signal sequence targeting the endosome, lysosome, autophagosome, or mitochondria, wherein the deleted ER signal sequence may be optionally replaced with an initiator methionine, wherein the initiator methionine may be optionally linked to an epitope tag.

The modified PDT or functional fragment thereof may comprise an about 15 to about 50 amino acid truncation relative to the wild-type PDI or functional fragment thereof.

In some embodiments, the modified PDI or functional fragment thereof is a PDI gene family member selected from the group of P4HB, ERp29, PDIA2, and TXNDC12. In some embodiments, the modified PDI or functional fragment thereof is not P4HB. In some embodiments, the modified PDI or functional fragment thereof is a human PDI.

The PDI family of proteins comprises about 20 members that differ in cell type expression and substrate specificities. The roles of the PDI family of enzymes help maintain proteostasis, here is thought that each PDI plays a distinct role in catalyzing folding of different substrates. See, generally Hiroyama et al., iScience 4, 102296, 23 Apr. 2021; doi:10.1016/j.isci.2021.102296. The luminal endoplasmic reticulum protein of 29 kDa (ERp29) regulates biosynthesis and trafficking of secretory proteins and transmembrane proteins, including cystic fibrosis transmembrane conductance regulator (CFTR), the epithelial sodium channel (ENaC), thyroglobulin, connexin 43 hemichannels, and proinsulin and appears to play a role in protein folding. See, e.g., Brecker et al, Front. Physiol., 8 Sep. 2020, Sec. Integrative Physiology; doi:10.3389/fphys.2020.574339. While PDI family member may not always comprise protein disulfide isomerase enzymatic activity prior to or subsequent to modification, in certain embodiments the PDI protein or fragment thereof retains of at least one of the biological activities of the protein, as detailed herein.

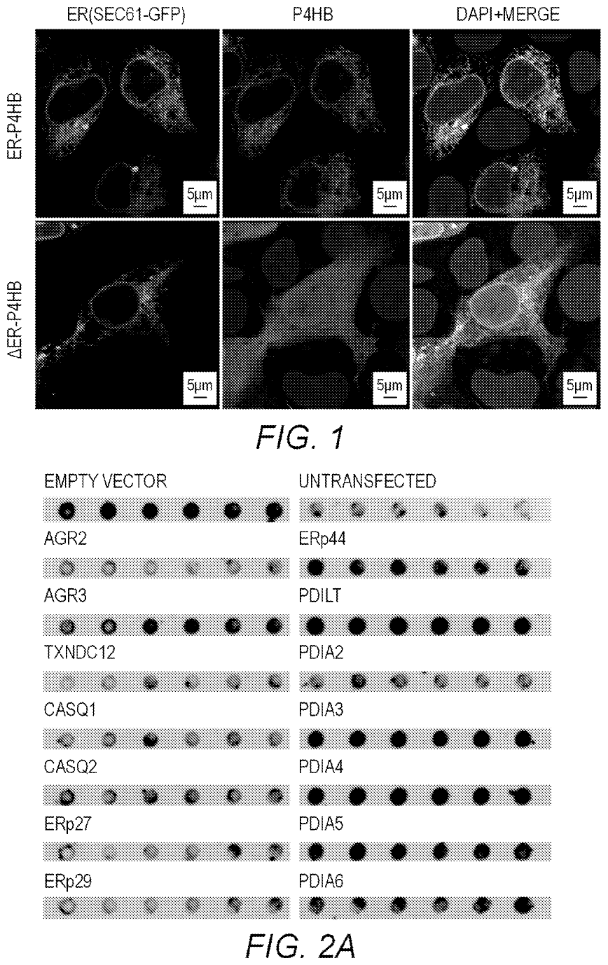

Protein disulfide isomerase family A member 2 (PDIA2) wild-type protein has an N-terminal ER signal, two catalytically active thioredoxin domains, two thioredoxin-like domains and a C-terminal retention sequence. The protein may comprise estradiol-binding activity, forming disulfide bonds, oxidase activity and/or reductase activity. Example domain organization of human PDI proteins is described in Lee, et al., BMB Rep. 2017 August; 50(8): 401-410.; doi:10.5473/BMBRep.2017.50.8.107; incorporated herein by reference in its entirety, with FIG. 1 and Table 1 incorporated herein by reference for domain organization, including ER-retention sequences.

Another aspect of the invention is a nucleic acid molecule encoding one or more of the modified PDIs or functional fragments thereof as taught herein. In one aspect, the nucleic acid molecule is operably linked to a promoter, wherein the promoter is optionally a brain-specific or brain-preferred promoter. In another aspect, the nucleic acid molecule is operably linked to a promoter, wherein the promoter is optionally a spinal cord-specific or spinal cord-preferred promoter. In another aspect, the nucleic acid molecule is operably linked to a promoter, wherein the promoter is optionally a muscle-specific or muscle-preferred promoter. Examples of suitable promoters include, without limitation, ChAT, HB9, Des, SynI, CAMKIIa, desmin, and UBEC. Tissue-specific promoters can be identified, for example using methods as described, for example, in Zheng and Baum, Methods Mol Biol. 2008; 434: 205-219; doi:10.1007/978-1-60327-248-3_13; see also Parambi et al., 2021 Oct 15, Mol Neurobiol. 2022; 59(1): 191-233, doi:10.1007/s12035-021-02555-y, incorporated herein by reference in its entirety, and specifically for its teaching of promoters, routes of administration and viral vector delivery.

Another aspect of the invention is a vector comprising one or more of the nucleic acid molecules as taught herein. In one aspect, the vector is one of the vectors described above, e.g., a viral vector, wherein the viral vector is optionally an adeno-associated viral vector. In an embodiment, the AAV is a naturally occurring AAV variant, e.g., AAV1, AAV2. In an embodiment, the AAV is a synthetic AAV capsid variant. In an aspect, the AAV is selected for tissue-specific delivery. AAV9 variants, for example AAV-PHP.B can be used for where desired to cross the blood brain barrier. AAV variants with reduced immunogenicity may also be utilized, and may comprise chimeric AAV, for example. AAV-DJ. Design strategies for AAV vectors may also be employed for the delivery of the modified PDI according to the present invention. See, e.g., Lee, et al. (2018) Adeno-associated virus (AAV) vectors: rational design strategies for capsid engineering. Curr. Opin. Biomed. Eng., 7, 58-63; see also Parambi et al., 2021 Oct 15, Mol Neurobiol. 2022; 59(1); 191-233, doi:10.1007/s12035-021-02555-y, incorporated herein by reference in its entirety, and specifically Table 1 for teachings of viral vectors.

A further aspect of the invention is a host cell comprising one or more of the modified PDIs or functional fragments thereof of the invention. In some embodiments, the host cell is an animal cell. In some embodiments, the animal cell is a human cell. In some embodiments, the host cell is an insect, yeast, or bacterial cell.

An additional aspect of the invention is a host cell comprising one or more of the nucleic acid molecules of the invention.

Another aspect of the invention is a host cell comprising one or more of the vectors of the invention.

Another aspect of the invention is a composition comprising one or more of the modified PDIs or functional fragment thereof, nucleic acid molecules, or vectors of the invention.

A further aspect of the invention is a pharmaceutical composition comprising one or more of the modified PDIs or functional fragment thereof, nucleic acid molecules, or vectors of the invention, and a pharmaceutically acceptable carrier.

Suitable carriers include, but are not limited to, salts, diluents. (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, anti-infective agents, suspending agents, wetting agents, viscosity modifiers, tonicity agents, stabilizing agents, and other components and combinations thereof. Suitable pharmaceutically acceptable carriers are preferably selected from materials which are generally recognized as safe (GRAS) and may be administered to an individual without causing undesirable biological side effects or unwanted interactions.

Suitable pharmaceutical carriers and their formulations are described in Remington's Pharmaceutical Sciences, 23rd ed. 2020, Academic Press. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions. Typically, the components of the formulation are dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol. In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers. In some embodiments, the formulation can be distributed or packaged in a liquid form, or alternatively, as a solid, obtained, for example by lyophilization of a suitable liquid formulation, which can be reconstituted with an appropriate carrier or diluent prior to administration. The pharmaceutical compositions comprise the modified PDIs or functional fragment thereof as taught herein, any one of the nucleic acid molecules as taught herein, or any one of the vectors as taught herein. The pharmaceutical compositions can be formulated for medical and/or veterinary use.

Another aspect of the invention is a method of treating a disease associated with TDP-43 aggregation in a subject in need thereof, the method comprising administering to the subject a therapeutically effective amount of one or more of the modified PDIs or functional fragments thereof, nucleic acid molecules, or vectors of the invention, thereby treating the disease.

Another aspect of the invention is a method of treating a disease associated with mutations in the TARDBP gene in a subject in need thereof, the method comprising administering to the subject a therapeutically effective amount of one or more of the modified PDIs or functional fragments thereof, nucleic acid molecules, or vectors of the invention, thereby treating the disease. The mutation may be any mutation or combination of mutations in the TARDBP gene associated with increased aggregation. Examples of mutations include, without limitation, K263E, N267S, G287S, G290A, S292N, G294V, G294A, G295S, G295R, G295C, G298S, M311V, A315E, A315T, A321V, A321G, Q331K, S332N, G335D, M337V, QQ434R, N345K, G348C, G348V, G348R, N352S, N352T, G357S, G357R, M359V, R361S, R361T, P363A, G368S, Y374X, G376D, N378D, N378S, S379C, W385G, N390D, N390S, S393L, A90V, D169G, or any combination thereof.

The disease to be treated that is associated with TDP-43 aggregation in a subject or associated with mutations in the TARDBP gene in a subject, as taught herein, may be any disease, disorder or condition now known or later identified to be associated with TDP-43 aggregation in a subject or associated with mutations in the TARDBP gene in a subject. In some embodiments, the disease, disorder, or condition is a neurodegenerative disease. The neurodegenerative disease to be treated may include, but is not limited to, amyotrophic lateral sclerosis (ALS), frontotemporal lobar degeneration (FTLD), Huntington's disease, Parkinson's disease, Alzheimer's disease, and/or prion disease. In some embodiments, the disease to be treated that is associated with TDP-43 aggregation in a subject or associated with mutations in the TARDBP gene in a subject, as taught herein, may be a muscle disease. The muscle disease to be treated may include, but is not limited to, inclusion body myositis.

The methods taught herein can improve a range of physical, mental, and emotional attributes of the treated subject. The subject can show an improvement in one or more symptoms of a neurodegenerative disease. Such improvements include, but are not limited to, improved physical abilities such as fine motor skills (e.g., writing and typing, grasping small objects, cutting, pointing, etc.), or gross motor skills (e.g., walking, balance, jumping, standing up, throwing); improved sensations such as decreased tingling and/or increased sensitivity in extremities, reduced sensation of muscle weakness or rigidity, and reduced tremors or pain; improved cognitive abilities such as increased alertness, reduced memory loss/improved memory recall, increased cognitive comprehension, improved speech and sleep, improved puzzle-solving abilities, increased focus; and improved behavioral performance such as decreased apathy, depression, agitation, or anxiety, and improved mood and general contentment.

In some embodiments, the methods treat or prevent a disease, disorder, or condition by reducing the rate of aggregation of TDP-43 in the subject (e.g., reducing the rate of formation of protein inclusions). In some embodiments, the methods treat a disease, disorder, or condition by reducing the amount of aggregate of TDP-43 in the subject (e.g., reducing the amount of protein inclusions). In some embodiments, the methods prevent aggregation of TDP-43 in the subject. Thus, the methods can reduce and/or prevent formation of pathological inclusions in cells of a subject. For instance, the methods can treat and/or prevent pathological phase separation and aggregation of one or more TDP-43 proteins.

In some embodiments, the methods can disaggregate existing protein aggregates. Thus, the methods can reduce the amount of existing protein aggregates prior to beginning the methods. This can be important for patients experiencing neurodegenerative disease symptoms, as such patients are likely to have existing protein aggregates. Disaggregation of existing aggregates can be, but need not necessarily be, in addition to prevention or reduction of further aggregate formation.

The methods can generate neuroprotective results when performed in a subject. As used herein, the term “neuroprotective” refers to maintaining or improving existing neurological function in the target neurological organ or tissue (e.g., nerve, spinal cord), or can refer to maintaining or improving the rate or overall amount of neuronal cell death in target neuronal cells. For example, “neuroprotective” can refer to slowing the rate of nerve tissue destruction, deterioration, or malfunction, slowing the rate of neuronal cell death, reducing the rate at which nerve conduction speed slows, etc. In some embodiments, the methods can generate at least 5%, at least 10%,6, at least 20%, or at least 25% or more neuroprotective improvement, as compared to a control.

Another aspect of the invention relates to a method of delivering any one of the modified PDIs or functional fragments thereof of the invention, the method comprising administering to the subject one or more of the modified PDIs or functional fragments thereof, nucleic acid molecules, or vectors of the invention, thereby delivering the modified PDI or functional fragment thereof to the subject, wherein the modified PDI or functional fragment thereof may be optionally delivered to the brain, spinal cord, and/or muscle tissue of the subject.

Another aspect of the invention relates to a method of disaggregating protein inclusions comprising TDP-43 in a subject, the method comprising administering to the subject one or more of the modified PDIs or functional fragments thereof, nucleic acid molecules, or vectors of the invention, thereby disaggregating protein inclusions comprising TDP-43 in the subject.

Another aspect of the invention relates to a method of restoring function of TDP-43 in a subject, the method comprising administering to the subject one or more of the modified PDIs or functional fragments thereof, nucleic acid molecules, or vectors of the invention, thereby restoring function of TDP-43 in the subject.

Another aspect of the invention relates to a method of inhibiting formation of protein inclusions comprising TDP-43 in a subject, the method comprising administering to the subject any one of the modified PDIs or functional fragments thereof, nucleic acid molecules, or vectors of the invention, thereby inhibiting formation of protein inclusions comprising TDP-43 in the subject.

The administering step of any one of the methods described herein can include at least one, at least two, at least three, at least four, at least five, at least six, at least seven, at least eight, at least nine, or at least ten dosages. The administering step can be performed before the subject exhibits disease symptoms (e.g., prophylactically), or during or after disease symptoms occur. The administering step can be performed prior to, concurrent with, or subsequent to administration of other agents to the subject. In some embodiments, the administering step is performed prior to, concurrent with, or subsequent to the administration of one or more additional diagnostic or therapeutic agents. In some embodiments, the methods comprise administering one or more additional modified PDIs or functional fragments thereof, one or more additional nucleic acid molecules, one or more additional vectors, or one or more additional host cells of the invention. In some embodiments, at least two, at least three, at least four, or at least five different modified PDIs or functional fragments thereof, nucleic acid molecules, vectors, or host cells of the invention are administered.

In some embodiments, a subsequent administration is provided at least one day after a prior administration, or at least two days, at least three days, at least four days, at least five days, or at least six days after a prior administration. In some embodiments, a subsequent administration is provided at least one week after a prior administration, or at least two weeks, at least three weeks, or at least four weeks after a prior administration. In some embodiments, a subsequent administration is provided at least one month, at least two months, at least three months, at least six months, or at least twelve months after a prior administration.

### Pharmaceutical Compositions

As a further aspect, the invention provides pharmaceutical formulations and methods of administering the same to achieve any of the therapeutic effects (e.g., treatment of tauopathy) discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

One embodiment of the invention is a composition including an isolated polynucleotide sequence encoding a modified PDI or functional fragment thereof, a plasmid or vector containing the isolated polynucleotide sequence, or a transfected cell containing the plasmid or vector or the isolated polynucleotide sequence and a suitable carrier, diluent, or excipient, and optionally a pharmaceutically acceptable carrier, diluent, or excipient. In one embodiment, the composition is in a form suitable for parenteral, oral, rectal, systemic, urogenital, topical, intravitreal, intraocular, otic, intranasal, dermal, sublingual, or buccal administration.

The modified PDI or functional fragment thereof of the invention can be formulated for administration in a pharmaceutical carrier in accordance with known techniques. See, e.g., Remington, The Science And Practice of Pharmacy (23rd Ed. 2020). In the manufacture of a pharmaceutical formulation according to the invention, the modified PDI or functional fragment thereof (including the physiologically acceptable salts thereof) is typically admixed with, inter alia, an acceptable carrier. The carrier can be a solid or a liquid, or both, and is preferably formulated with modified PDI or functional fragment thereof as a unit-dose formulation, for example, a tablet, which can contain from 0.01 or 0.5% to 95% or 99% by weight of the modified PDI or functional fragment thereof. One or more modified PDI or functional fragment thereof can be incorporated in the formulations of the invention, which can be prepared by any of the well-known techniques of pharmacy.

A further aspect of the invention is a method of treating subjects in vivo, comprising administering to a subject a pharmaceutical composition comprising a modified PDI or functional fragment thereof of the invention in a pharmaceutically acceptable carrier, wherein the pharmaceutical composition is administered in a therapeutically effective amount. Administration of the modified PDI or functional fragment thereof of the present invention to a human subject or an animal in need thereof can be by any means known in the art for administering compounds.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound which is being used. In an embodiment, administration may be direct delivery to the cerebrospinal fluid(CSF) via intrathecal delivery, or administration utilizing delivery systems that can cross the blood brain barriers, e.g., via AAV vectors such as AAV9. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site, or intramuscular administration with muscle specific promoters). In some embodiments, the formulation can be delivered locally to ischemic tissue. In certain embodiments, the formulation can be a slow release formulation, e.g., in the form of a slow release depot.

For injection, the carrier will typically be a liquid, such as sterile pyrogen-free water, pyrogen-free phosphate-buffered saline solution, bacteriostatic water, or Cremophor EL[R](BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain antioxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

The amount of the disclosed compositions administered to a subject will vary from subject to subject, depending on the nature of the disclosed compositions and/or formulations, the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to reduce protein inclusions or to improve a symptom of a neurodegenerative disease). The dosage should not be so large as to outweigh benefits by causing extensive or severe adverse side effects, such as unwanted cross-reactions, anaphylactic reactions, and the like, although some adverse side effects may be expected. The dosage can be adjusted by the individual clinician in the event of any counterindications. Generally, the disclosed compositions and/or formulations are administered to the subject at a dosage of active component(s) ranging from 0.1 mg/kg body weight to 100 g/kg body weight. In some embodiments, the disclosed compositions and/or formulations are administered to the subject at a dosage of active component(s) ranging from 1 mg/kg to 10 g/kg, from 10 mg/kg to 1 g/kg, from 10 mg/kg to 500 mg/kg, from 10 mg/kg to 100 mg/kg, from 10 mg/kg to 10 mg/kg, from 10 mg/kg to 1 mg/kg, from 10 mg/kg to 500 mg/kg, or from 10 mg/kg to 100 mg/kg body weight. Dosages above or below the range cited above may be administered to the individual subject if desired. The compositions can be administered in any herein disclosed pharmaceutical composition comprising a pharmaceutically acceptable carrier.

### Non-Limiting Embodiments of the Invention Include the Following

Embodiment 1. A modified protein disulfide isomerase (PDI) or a functional fragment thereof, wherein the modified PDI or functional fragment thereof comprises a deletion of an N-terminal endoplasmic reticulum (ER) signal sequence from a wild-type PDT or functional fragment thereof, wherein the PDT is not P4HB.
Embodiment 2. The modified PDI or functional fragment thereof of Embodiment 1, wherein about 15 to about 50 amino acid residues are deleted from the N-terminus of the wild-type PDI or functional fragment thereof.
Embodiment 3. The modified PDI or functional fragment thereof of Embodiment 1 or 2, wherein the deleted ER signal sequence from the N-terminus is replaced with an initiator methionine.
Embodiment 4. The modified PDI or functional fragment thereof of Embodiment 1 or 2, wherein the deleted ER signal sequence from the N-terminus is replaced with an initiator methionine linked to an epitope tag.
Embodiment 5. A modified protein disulfide isomerase (PDI) or a functional fragment thereof, wherein the modified PDI or functional fragment thereof comprises a deletion of a C-terminal endoplasmic reticulum (ER) signal sequence from a wild-type PDI or functional fragment thereof.
Embodiment 6. A modified protein disulfide isomerase (PDI) or a functional fragment thereof, wherein the modified PDI or functional fragment thereof comprises a deletion of an endoplasmic reticulum (ER) signal sequence from a wild-type PDI or functional fragment thereof and further comprises a nuclear localization signal (NLS).
Embodiment 7. The modified PDT or functional fragment thereof of any one of Embodiments 1-6, wherein the modified PDI is a human PDI.
Embodiment 8. The modified PDT or functional fragment thereof of any one of Embodiments 1-7, wherein the modified PDI or functional fragment thereof is a PDI gene family member selected from the group of ERp29, PDIA2, and TXNDC12.
Embodiment 9. The modified PDI or functional fragment thereof of any one of Embodiments 5-7, wherein the modified PDI or functional fragment thereof is a PDI gene family member selected from the group of P4HB, ERp29, PDIA2, and TXNDC12.
Embodiment 10. The modified PDI or functional fragment thereof of Embodiment 9, wherein the modified PDI or functional fragment thereof is the PDI gene family member P4HB.
Embodiment 11. The modified PDI or functional fragment thereof of Embodiment 8 or 9, wherein the modified PDI or functional fragment thereof is the PDI gene family member ERp29.
Embodiment 12. The modified PDI or functional fragment thereof of Embodiment 8 or 9, wherein the modified PDT or functional fragment thereof is the PDI gene family member PDTA2.
Embodiment 13. The modified PDI or functional fragment thereof of Embodiment 8 or 9, wherein the modified PDI or functional fragment thereof is the PDT gene family member TXNDC12.
Embodiment 14. A nucleic acid molecule encoding the modified PDI or functional fragment of any one of Embodiments 1-13.
Embodiment 15. The nucleic acid molecule of Embodiment 14, operably linked to a promoter.
Embodiment 16. The nucleic acid molecule of Embodiment 15, wherein the promoter is a brain-specific or brain-preferred promoter.
Embodiment 17. The nucleic acid molecule of Embodiment 15, wherein the promoter is a spinal cord-specific or spinal cord-preferred promoter.
Embodiment 18. The nucleic acid molecule of Embodiment 15, wherein the promoter is a muscle-specific or muscle-preferred promoter.
Embodiment 19. A vector comprising the nucleic acid molecule of any one of Embodiments 14-18.
Embodiment 20. The vector of Embodiment 19, which is a viral vector.
Embodiment 21. The vector of Embodiment 20, which is an adeno-associated virus vector.
Embodiment 22. A host cell comprising the modified PDI or functional fragment of any one of Embodiments 1-13.
Embodiment 23. A host cell comprising the nucleic acid molecule of any one of Embodiments 14-18.
Embodiment 24. A host cell comprising the vector of any one of Embodiments 19-21.
Embodiment 25. A composition comprising the modified PDI or functional fragment thereof of any one of Embodiments 1-13, the nucleic acid of any one of Embodiments 14-18, the vector of any one of Embodiments 19-21, or the host cell of any one of Embodiments 22-24.
Embodiment 26. A pharmaceutical composition comprising the modified PDI or functional fragment thereof of any one of Embodiments 1-13, the nucleic acid of any one of Embodiments 14-18, the vector of any one of Embodiments 19-21, or the host cell of any one of Embodiments 22-24 and a pharmaceutically acceptable carrier.
Embodiment 27. A method of treating a disease associated with TAR DNA-binding protein 43 (TDP-43) aggregation in a subject in need thereof, the method comprising administering to the subject a therapeutically effective amount of a modified protein disulfide isomerase (PDT) or a functional fragment thereof wherein the modified PDT or functional fragment thereof comprises a deletion of an endoplasmic reticulum (ER) signal sequence from a wild-type PDI or functional fragment thereof, thereby treating the disease.
Embodiment 28. The method of Embodiment 27, wherein administering a modified PDI or a functional fragment thereof comprises administering to the subject the modified PDI or functional fragment thereof of any one of Embodiments 1-13, the nucleic acid of any one of Embodiments 14-18, the vector of any one of Embodiments 19-21, the host cell of any one of Embodiments 22-24, the composition of Embodiment 25, or the pharmaceutical composition of Embodiment 26.
Embodiment 29. A method of treating a disease associated with mutations in the TARDBP gene in a subject in need thereof, the method comprising administering to the subject a therapeutically effective amount of a modified protein disulfide isomerase (PDI) or a functional fragment thereof wherein the modified PDI or functional fragment thereof comprises a deletion of an endoplasmic reticulum (ER) signal sequence from a wild-type PDI or functional fragment thereof, thereby treating the disease.
Embodiment 30. The method of Embodiment 29, wherein administering a modified PDI or a functional fragment thereof comprises administering to the subject the modified PDI of functional fragment thereof of any one of Embodiments 1-13, the nucleic acid of any one of Embodiments 14-18, the vector of any one of Embodiments 19-21, the host cell of any one of Embodiments 22-24, the composition of Embodiment 25, or the pharmaceutical composition of Embodiment 26.
Embodiment 31. The method of any one of Embodiments 27-30, wherein the disease is a neurodegenerative disease.
Embodiment 32. The method of Embodiment 31, wherein the neurodegenerative disease is amyotrophic lateral sclerosis (ALS).
Embodiment 33. The method of Embodiment 31, wherein the neurodegenerative disease is frontotemporal lobar degeneration (FTLD).
Embodiment 34. The method of Embodiment 31, wherein the neurodegenerative disease is Huntington's disease.
Embodiment 35. The method of Embodiment 31, wherein the neurodegenerative disease is Parkinson's disease.
Embodiment 36. The method of Embodiment 31, wherein the neurodegenerative disease is Alzheimer's disease.
Embodiment 37. The method of Embodiment 31, wherein the neurodegenerative disease is prion disease.
Embodiment 38. The method of any one of Embodiments 27-30, wherein the disease is inclusion body myositis.
Embodiment 39. A method of delivering a modified PDI or functional fragment thereof to a subject, the method comprising administering to the subject a modified protein disulfide isomerase (PDI) or a functional fragment thereof wherein the modified PDI or functional fragment thereof comprises a deletion of an endoplasmic reticulum (ER) signal sequence from a wild-type PDI or functional fragment thereof, thereby delivering the modified PDI or functional fragment thereof.
Embodiment 40. The method of Embodiment 40, wherein administering a modified PDI or a functional fragment thereof comprises administering to the subject the modified PD1 of functional fragment thereof of any one of Embodiments 1-13, the nucleic acid of any one of Embodiments 14-18, the vector of any one of Embodiments 19-21, the host cell of any one of Embodiments 22-24, the composition of Embodiment 25, or the pharmaceutical composition of Embodiment 26.
Embodiment 41. The method of Embodiment 39 or 40, wherein the modified PDI or functional fragment thereof is delivered to the brain of the subject.
Embodiment 42. The method of Embodiment 39 or 40, wherein the modified PDI or functional fragment thereof is delivered to the spinal cord of the subject.
Embodiment 43. The method of Embodiment 39 or 40, wherein the modified PDI or functional fragment thereof is delivered to muscle tissue of the subject.
Embodiment 44. A method of disaggregating protein inclusions comprising TDP-43 in a subject, the method comprising administering to the subject a modified protein disulfide isomerase (PDI) or a functional fragment thereof wherein the modified PDI or functional fragment thereof comprises a deletion of an endoplasmic reticulum (ER) signal sequence from a wild-type PDI or functional fragment thereof, thereby disaggregating protein inclusions comprising TDP-43.
Embodiment 45. The method of Embodiment 44, wherein administering a modified PDT or a functional fragment thereof comprises administering to the subject the modified PDT of functional fragment thereof of any one of Embodiments 1-13, the nucleic acid of any one of Embodiments 14-18, the vector of any one of Embodiments 19-21, the host cell of any one of Embodiments 22-24, the composition of Embodiment 25, or the pharmaceutical composition of Embodiment 26.
Embodiment 46. A method of inhibiting formation of protein inclusions comprising TDP-43 in a subject, the method comprising administering to the subject a modified protein disulfide isomerase (PDI) or a functional fragment thereof wherein the modified PDI or functional fragment thereof comprises a deletion of an endoplasmic reticulum (ER) signal sequence from a wild-type PD1 or functional fragment thereof, thereby inhibiting formation of protein inclusions comprising TDP-43.
Embodiment 47. The method of Embodiment 46, wherein administering a modified PDI or a functional fragment thereof comprises administering to the subject the modified PD1 of functional fragment thereof of any one of Embodiments 1-13, the nucleic acid of any one of Embodiments 14-18, the vector of any one of Embodiments 19-21, the host cell of any one of Embodiments 22-24, the composition of Embodiment 25, or the pharmaceutical composition of Embodiment 26.
Embodiment 48. A method of restoring function of TDP-43 in a subject, the method comprising administering to the subject a modified protein disulfide isomerase (PDI) or a functional fragment thereof wherein the modified PDI or functional fragment thereof comprises a deletion of an endoplasmic reticulum (ER) signal sequence from a wild-type PDI or functional fragment thereof, thereby restoring function of TDP-43.
Embodiment 49. The method of Embodiment 48, wherein administering a modified PDI or a functional fragment thereof comprises administering to the subject the modified PDI of functional fragment thereof of any one of Embodiments 1-13, the nucleic acid of any one of Embodiments 14-18, the vector of any one of Embodiments 19-21, the host cell of any one of Embodiments 22-24, the composition of Embodiment 25, or the pharmaceutical composition of Embodiment 26.
Embodiment 50. The method of any one of Embodiments 27-49, wherein the modified PDI of functional fragment thereof is delivered to the cytoplasm of cells in the subject, delivered to the nucleus of cells in the subject, or a combination thereof.

Having described the present invention, the same will be explained in greater detail in the following examples, which are included herein for illustration purposes only, and which are not intended to be limiting to the invention.

### EXAMPLES

### Example 1: Ablating ER-Signal Sequence to Target PDI Expression to Cytosol

Selective modification of Protein Disulfide Isomerase. The modified P4HB or a functional fragment thereof contains a novel modification that targets cellular expression to the cytoplasm and nucleoplasm. Specifically in this example, the N-terminal ER signal sequence, containing the first 17 amino acid residues have been excised and replaced with an initiator methionine-flanked 3× FLAG epitope tag. Ablation of this ER signal sequence prevents co-translational insertion of P4HB into the ER membrane and thus leads to cytosolic expression. Additionally, this choice of modification permits nuclear localization of P4HB otherwise not observed with overexpression of the native isoform. HEK293T cells were transfected with either native P4HB (ER-P4HB) or ΔER-P4HB, plus a GFP-tagged Sec61 fusion protein to serve as an ER counterstain. Cells were fixed, probed for P4HB, counterstained with the nuclear stain DAPI and imaged using a Zeiss 8000 laser scanning confocal microscope under 63× oil immersion objective. See FIG. 1. Deletion of the N-terminal ER signal sequence resulted in P4HB localization to the cytoplasm and nucleoplasm. This is visualized by counterstaining the ER with GFP-Sec61. Alack of co-localization between P4HB and Sec61 was noted, as well as increased co-localization between DAPI and P4HB following selective mutation.

### Example 2: Screening Platform Identifies PDI Candidates that Reduce TDP-43 Pathology

The Protein Disulfide Isomerase family consists of 15 gene members with varying degrees of chaperone and/or isomerase activity. An unbiased screen to identify candidates that most effectively attenuate TDP-43 pathology was performed. The panel of candidates included all PDI members (anterior gradient 2 (AGR2), AGR3, TXNDC12 (also referred to as AGR1), CASQ1, CASQ2, ERp27, ERp29, ERp44, PDILT, PDIA2, PDIA3, PDIA4, PDIA5, and PDIA6) that underwent identical ER-modification process for cytoplasmic expression, wherein the N-terminal ER signal sequence, containing the first several amino acid residues as indicated in Table 1 have been excised and replaced with an initiator methionine-flanked 3× FLAG epitope tag.

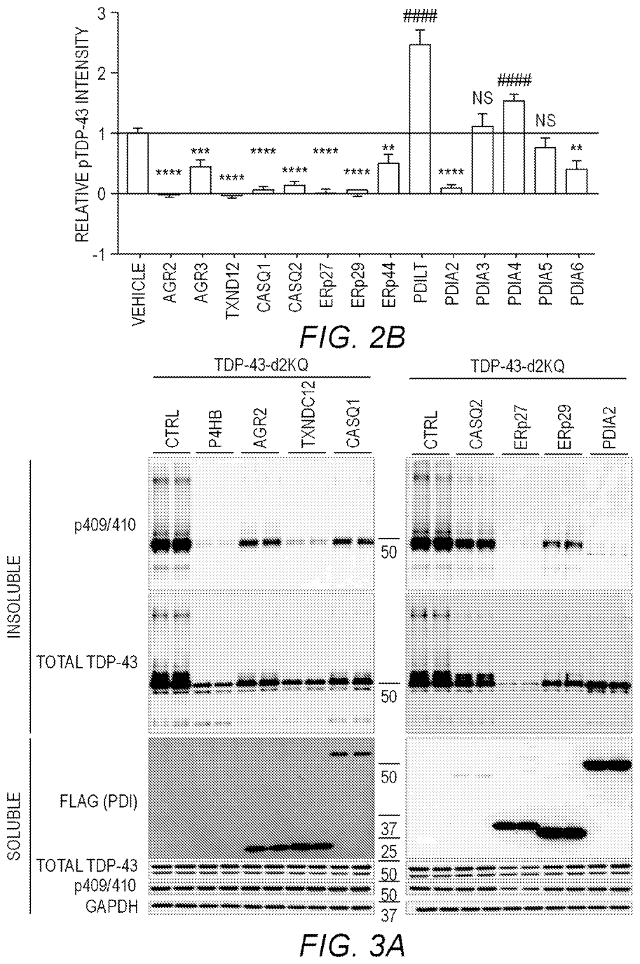

Using a multi-well assay, HEK293A cells were transfected with a TDP-43-dNLS-2KQ variant (K145/192Q mutations to de-stabilize interactions between TDP-43, encouraging multimerization and mutation to the NLS to induce cytoplasmic mislocalization of the multimerizing complexes, where they then form disulfide cross-linked aggregates) and each PDI candidate or control as indicated. Cells were lysed in a chaotropic buffer, and protein lysates were adsorbed onto a nitrocellulose membrane in biological sextuplets. The membrane was probed with an antibody specific for phosphorylated TDP-43 at serine 409/410. See FIG. 2A.

Phosphorylated TDP-43 signal was normalized relative to total TDP-43 input. Modified PDI candidates were refined to include those that reduced more than 83% of phosphorylated TDP-43 signal for further biochemical characterization (including modified candidates of the PDI family member P4HB, AGR2, TXNDC12, CASQ1, CASQ2, ERp27, ERp29, and PDIA2). See FIG. 2B. These data reveal several modified candidates which alleviate TDP-43 aggregation extensively (>80%), some which have an intermediate affect, and some which exacerbate aggregation, as evidenced by changes to normalized phosphor-TDP-43 intensity. Candidates that proved affective at removing >80% of TDP-43 pathology were selected for downstream biochemical characterization.

### Example 3: P4HB (PDIA1) is Most Efficient at Reducing TDP-43 Pathology

Biochemical validation of modified PDT candidates identified from the multi-well assay of Example 2 revealed modified P4HB as most effective at ameliorating TDP-43 pathology. Candidates that reduced greater than or equal to 83% of pS409/410 TDP-43 signal were subject to solubility fractionation and immunoblot (including modified candidates of the PDT family member P4HB, AGR2, TXNDC12, CASQ1, CASQ2, ERp27, ERp29, and PDIA2).

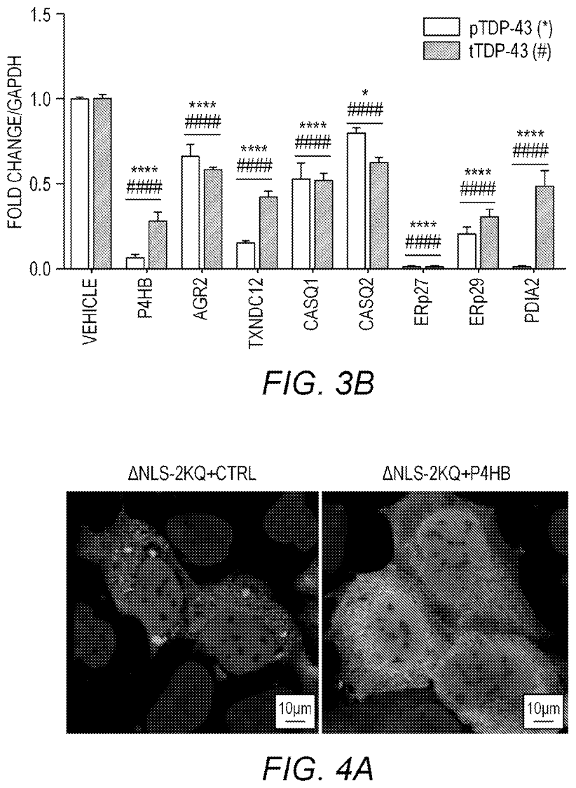

HEK293 cells were transfected with TDP-43-dNLS-2KQ plus a modified PDI candidate from the group of P4HB, AGR2, TXNDC12, CASQ1, CASQ2, ERp27, ERp29, and PDIA2 or control vector expressing 3×FLAG-HA. Cells were lysed in radioimmunoprecipitation assay buffer, sonicated, and centrifuged at 20,000 RCF (relative centrifugal field). The insoluble TDP-43-containing pellet was resuspended in 8M urea followed by sonication and centrifugation at 20,000 RCF. Lysates were prepared for reducing SDS-PAGE followed by immunoblotting for: pS409/410, total TDP-43, FLAG, and GAPDH. See FIG. 3A. Refined candidates abated the formation of insoluble phosphorylated and total TDP-43 to variable extents (P4HB, AGR2, TXNDC12, CASQ1, CASQ2, ERP29, PDIA2). Densitometric normalization and quantification is performed in 3B.

The insolubility levels of TDP-43 by PD1 family member were analyzed with densitometry. The parameters for the densitometry measurements were as follows: 50 kDa band, equal sampling area across all conditions with background subtraction, normalized to GAPDH. Relative reduction in insoluble pS409/410 and total TDP-43 were plotted as mean+/−standard deviation. Alpha=0.05, calculated using Two-way ANOVA with Tukey's multiple comparisons test. See FIG. 3B. Data here refines candidates from the initial screen to those that can prevent aggregation, insoluble conversion, and pathological phosphorylation of TDP-43. Of note, P4HB appears to be most effective.

### Example 4: Clearance of Cytosolic TDP-43 Pathology is Thioredoxin Dependent

P4HB clearance of cytosolic TDP-43 pathology is thioredoxin dependent and occurs through physical interaction with P4HB.

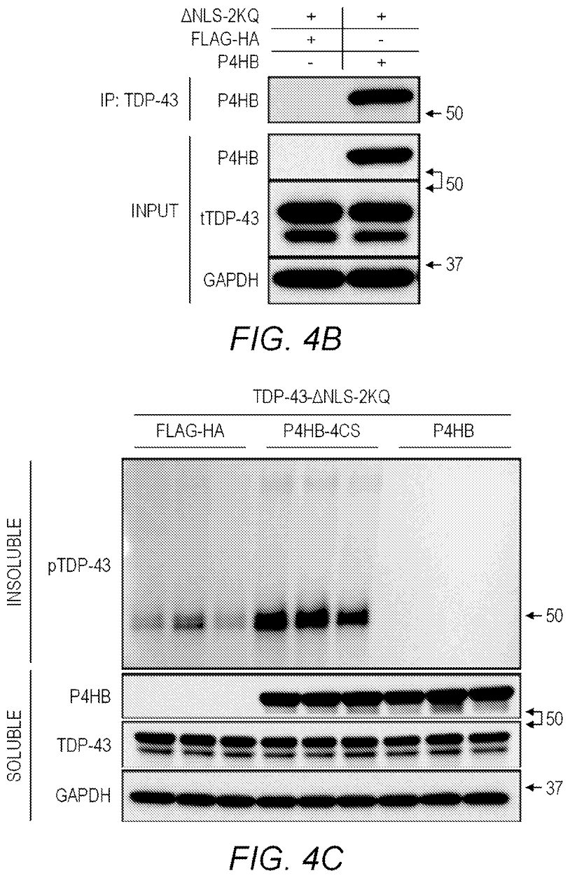

HEK293 cells were transfected with TDP-43-dNLS-2KQ and control (3×FLAG-HA) or modified P4HB. Cells were fixed, probed for TDP-43 and P4HB, and imaged using laser scanning confocal microscopy. Cells containing modified P4HB were void of cytoplasmic TDP-43 inclusions. See FIG. 4A.

HEK293 cells were transfected with TDP-43-dNLS-2KQ and control (3×FLAG-HA) or modified P4HB. TDP-43 was immunoprecipitated and subject to immunoblotting for P4HB. See FIG. 4B. A physical interaction was identified between TDP-43 and modified (ER signal-seq-deleted) P4HB relative to pcDNA3.1-FLAG-HA control vector, by co-immunoprecipitation.

HEK293 cells were transfected with TDP-43-dNLS-2KQ and control vector (3×FLAG-HA), modified P4HB (ER ablation), or modified P4HB containing ablated thioredoxin motifs (4CS). Cells were subject to solubility fractionation as above and probed for pS409/410. Here, a return of pathological TDP-43 was identified, and a seemingly dominant-negative phenotype when the thioredoxin motifs are ablated. This purports that modified P4HB requires thioredoxin catalysis to disaggregate cytosolic TDP-43 pathology. See FIG. 4C.

### Example 5: Clearance of Cytosolic TDP-43 Pathology is Thioredoxin Dependent

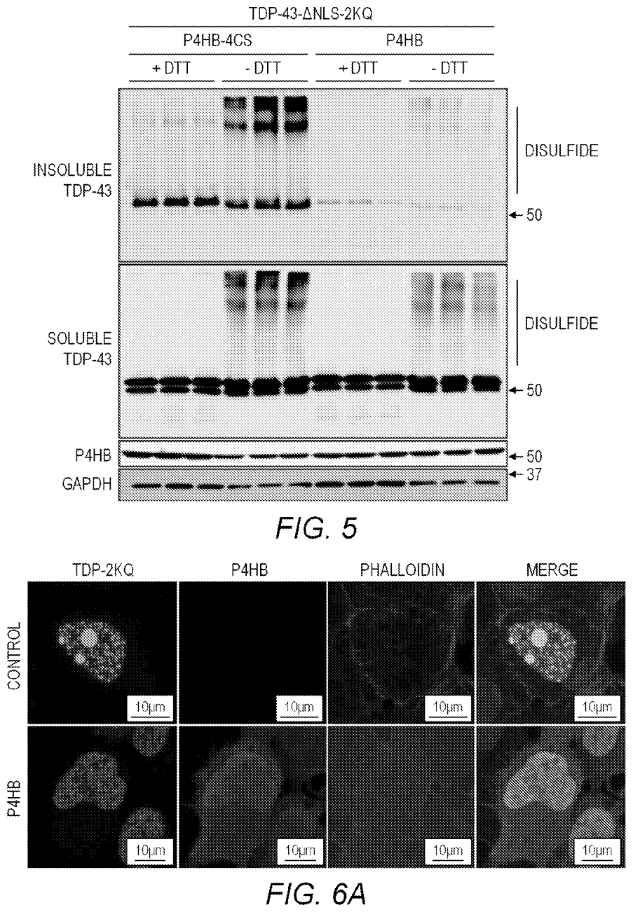

Cytosolic TDP-43 pathology clearance is thioredoxin dependent. Non-reducing PAGE of cells co-expressing TDP-43-dNLS-2KQ and functional (ER signal ablated) or thioredoxin-null engineered (ER signal ablated/4CS) P4HB were compared. Increased disulfide-linked multimers were present with thioredoxin-null P4HB, validating previous studies that cytosolic TDP aggregates are stabilized by disulfide linkages and confirms a thioredoxin-dominant clearance mechanism of TDP-43 disaggregates. See FIG. 5.

### Example 6: Engineered P4HB Accesses the Nucleus and Abrogates Nascent TDP-43 Condensates Prior to Cytoplasmic Mislocalization

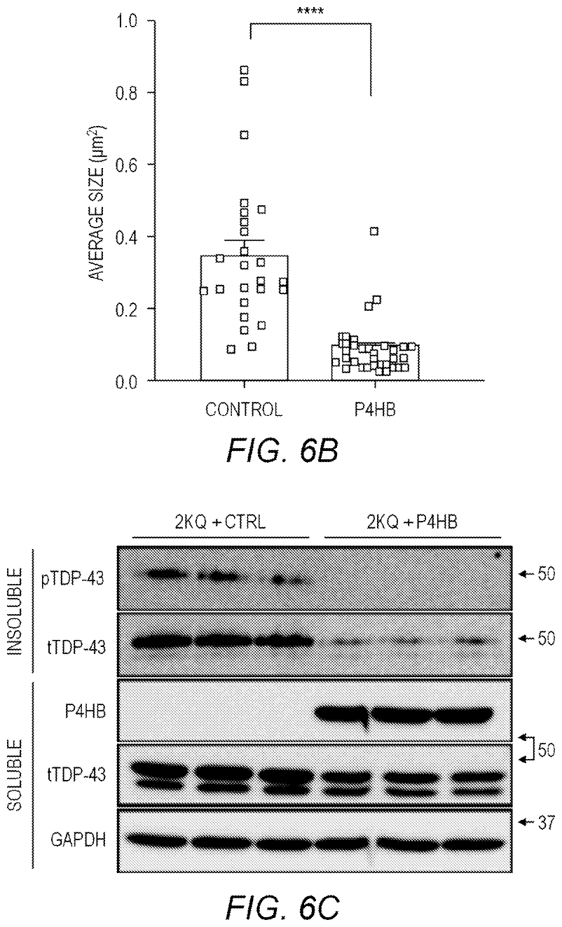

HEK293 cells were transfected with a nuclear-localized, aggregation prone TDP-43 variant (TDP-43-2KQ) which resulted in the formation of phase separated liquid condensates in the nuclear compartment. The ability of modified P4HB to gain access to the nucleoplasm led to a significant reduction in the average size of nuclear TDP-43 foci. This reduction in the average size of nuclear TDP-43 foci suggests that in addition to targeting established cytoplasmic TDP pathology, modified P4HB can target early, pre-pathological species prior to cytoplasmic mislocalization. Images represent the mean area of TDP foci per condition. Images were acquired from 50 cells per condition on a Zeiss 8000 Laser Scanning Confocal Microscope and analyzed using ImageJ. See FIG. 6A.

The average size of nuclear TDP-43 foci was larger in the control (3×FLAG-HA) than in the presence of modified P4HB. See FIG. 6B.

Biochemical fractionation confirmed the confocal microscopy findings that are shown in FIG. 6A that the presence of modified P4HB reduces the extent of insoluble, phosphorylated, nuclear TDP-43 pathology. See FIG. 6C. HEK293A cells were transfected with nuclear-resident, mutant TDP-43-2KQ with either modified (ER signal-sequence deleted) P4HB or pcDNA3.1-FLAG-HA control. Cells were lysed in radioimmunoprecipitation assay buffer, sonicated, and centrifuged at 20,000 RCF. The insoluble TDP-43-containing pellet was resuspended in 8M urea followed by sonication and centrifugation at 20,000 RCF. Lysates were prepared for reducing SDS-PAGE followed by immunoblotting for: pS409/410, total TDP-43, FLAG, and GAPDH. Data here indicate a significant decrease in insoluble, phos-TDP-43 in the presence of modified P4HB, confirming a functional impact of modified P4HB's ability to translocate to the nucleus and rescue early TDP-43 pathology prior to cytoplasmic mislocalization.

### Example 7: P4HB Prevents TDP-43 Co-Aggregation with Nuclear Pore Complex-153

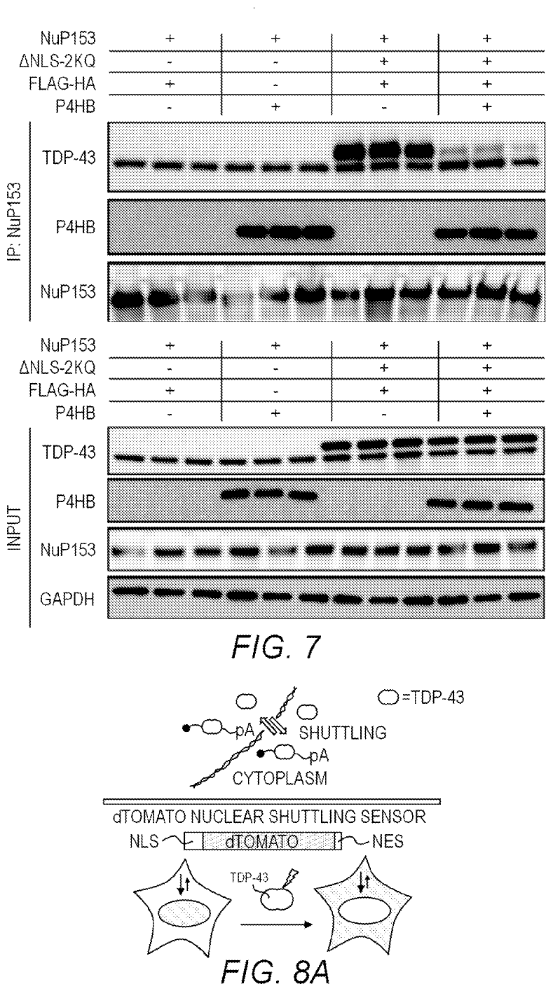

Modified (ER signal ablated) P4HB prevents rescues co-aggregation between cytoplasmic TDP-43 and NuP153. HEK293 cells were transfected as indicated. Cells were lysed and subject to co-immunoprecipitation using anti-Nup153 capture beads. Precipitants were analyzed by western blot probing for TDP-43 and P4HB. This western blot demonstrates Nup153 co-aggregation with cytoplasmic TDP-43, and for the first time demonstrates an ability for modified P4HB to prevent co-aggregation of pathological TDP-43 and Nup153. This suggests modified P4HB can restore native cellular function of TDP-43 and its associated nuclear processes. See FIG. 7.

### Example 8: P4HB Rescues TDP-Induced Nuclear Shuttling Defects

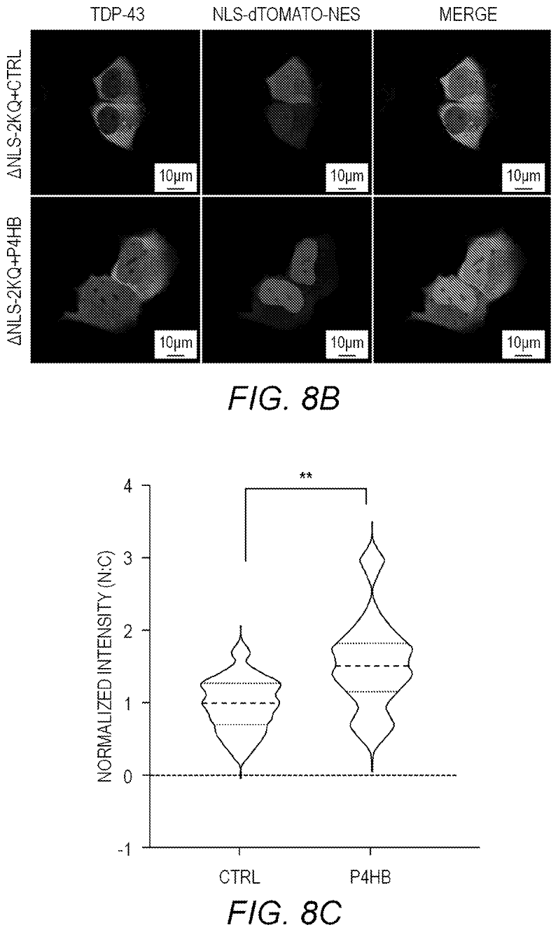

Modified (ER signal ablated) P4HB rescues nuclear-cytoplasmic shuttling defects that result from TDP-43 aggregation. A key function of TDP-43 is iterative shuttling of protein and mRNA complexes into and out of the nucleus. A nuclear-cytoplasmic shuttling sensor that consists of a dTomato fluorochrome appended to a nuclear localization- and nuclear export-sequence was used (NLS-dTomato-NESO (FIG. 8A)). TDP-43-dNLS-2KQ was co-expressed with either control (3×FLAG-HA) or modified P4HB in the presence of the shuttling sensor (FIG. 8B). The ratio of nuclear:cytoplasmic fluorescence intensity was calculated to determine nuclear shuttling efficacy. Modified P4HB increased shuttling performance in cells expressing pathological TDP-43 relative to control. Violin plots represent 30 cells per condition imaged using a Zeiss 8000 laser scanning confocal microscope, analyzed in ImageJ. See FIG. 8C. In the presence of modified P4HB, the quantified nuclear:cytoplasmic ratio of dTomato increases relative to cells expressing pcDNA3.1-FLAG-HA control, suggesting a P4HB-mediated corrective property to TDP-43 nucleo-cytoplasmic shuttling.

### Example 9: P4HB Rescues Transcriptional Dysfunction Associated with Pathological TDP-43

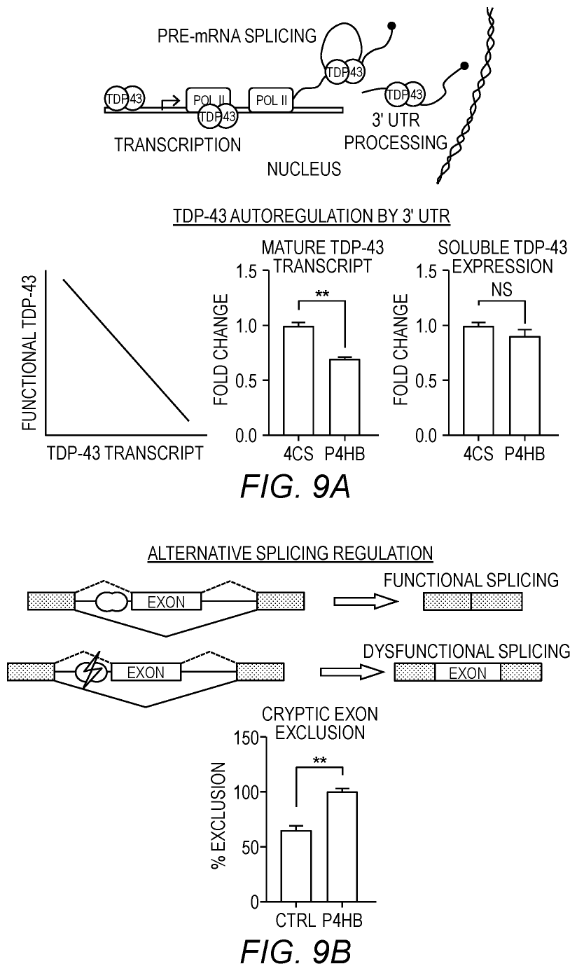

TDP-43 is tightly regulated in the mammalian cell and has autoregulatory properties by engaging its own 3′-UTR for degradation in response to accumulation of functional (soluble) TDP-43 protein. Changes to mature TDP-43 transcripts were assessed by RT-qPCR in cells expressing TDP-43-dNLS-2KQ and either functional (ER signal ablated) or thioredoxin-null (ER signal ablated/4CS) P4HB. A significant decline in mature TARDBP transcript in cells expressing functional TDP-43 as compared to thioredoxin-null was observed, with no significant change in soluble TDP-43 protein. This suggests that in the presence of functional P4HB, TDP-43 is stabilized and maintains partial functionality in contrast with expression of thioredoxin-null P4HB. See FIG. 9A.

To validate modified P4HB-mediated rescue of transcriptional regulation, a minigene splicing reporter assay was employed. Cells were transfected with TDP-43-dNLS-2KQ and control (3×FLAG-HA) or modified P4HB, in the presence of a CFTR Exon 9 bait transcript. Modified TDP-43 splices and excises cryptic Exon 9 resulting in a small cDNA amplicon by RT-PCR Increased TDP-43 splicing capability was identified when modified P4HB was present relative to mock controls. See FIG. 9B.

### Example 10: Tissue Specific Delivery of P4HB Rescues TDP Pathology in Model of Inclusion Body Myositis

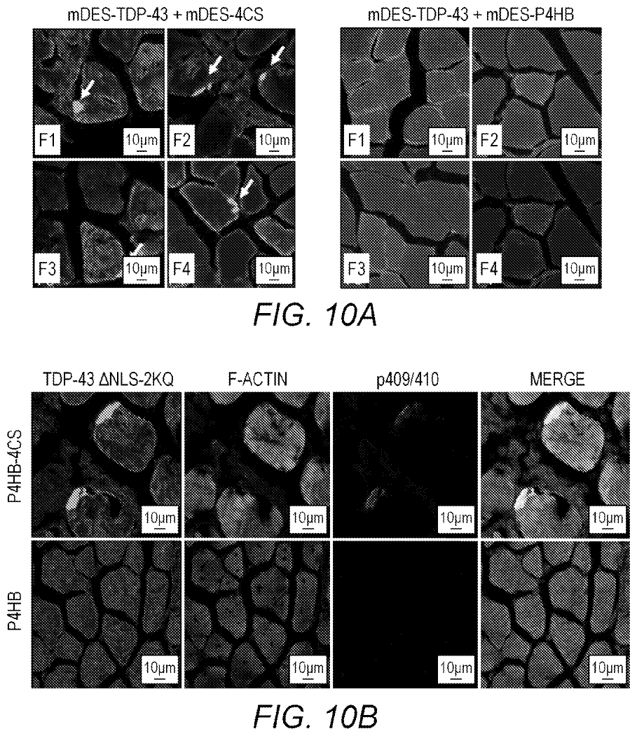

GFP-TDP-43-dNLS-KQ was expressed with thioredoxin-null (ER signal ablated/4CS) P4HB or modified P4HB (ER signal ablated) in adult murine tibialis anterior (TA) muscle under skeletal muscle-specific desmin promoter. TA muscle was removed, sectioned, and immunolabeled for TDP-43. See FIG. 10A.

Fewer myofibrils positive for p409/410+ puncta are noted in the muscle where modified P4HB was present. See FIG. 10B.

### Example 11: ER-P4HB Variants Upregulate Unfolded Protein Response (UPR) Pathway X-Box Binding Protein 1 (XBP1)

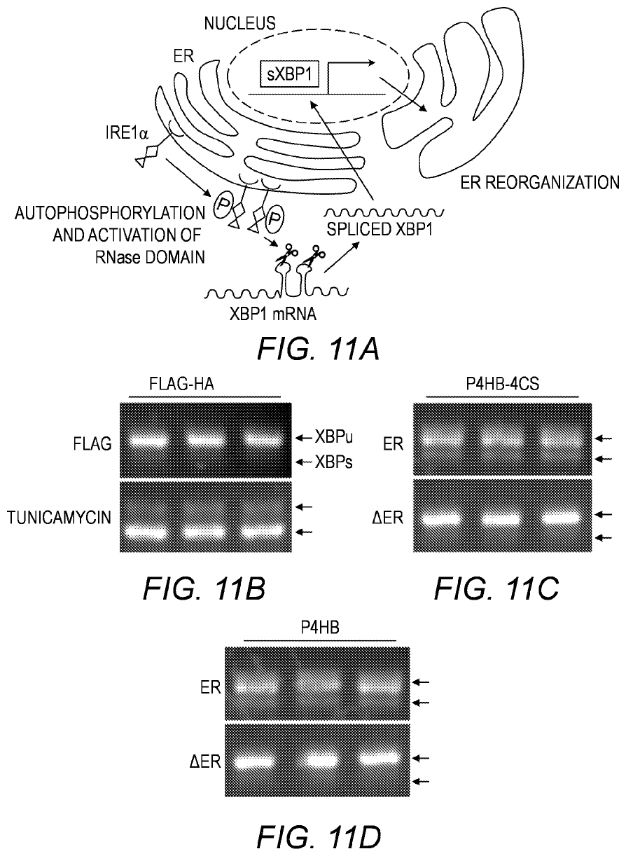

The XBP1 UPR pathway is depicted in FIG. 11A. HEK293 were transfected with TDP-43-dNLS-2KQ with empty vector, ER-localized (wt)P4HB, ER-localized thioredoxin null P4HB, liberated P4HB, and liberated thioredoxin null P4HB. For a positive control of ER stress, 1.0 μM of tunicamycin was added to transfected cells. RNA was extracted followed by cDNA conversion and RT-PCR of XBP1 alternative splice variants. Under instances of increased ER stress, there is an increase in XBP1 splicing (XBPs), which is a precursor for transcriptional activation and upregulation of UPR/ERAD regulators such as BiP. FIGS. 11B-11D show liberation of ER-resident P4HB avoids upregulation of ER-stress and unfolded-protein response (UPR) pathway, while maximizing TDP-43 disaggregation. It is seen in the immunoblots that regardless of thioredoxin potential, overexpression of modified P4HB prevents induction of an ER-stress response, further supporting the utility in use of this modified protein for clearing TDP-43 pathology. (FIG. 11B-11D).

### Example 12: ER-P4HB Variants Upregulate XBP1 Target, UPR Chaperone BiP

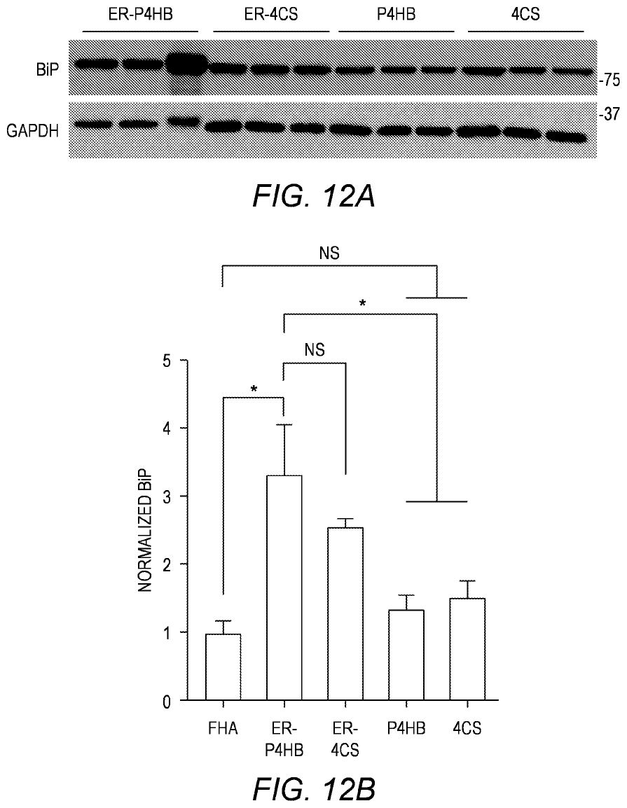

HEK293 were transfected with TDP-43-dNLS-2KQ with empty vector, ER-localized (wt) P4HB, ER-localized thioredoxin null P4HB, liberated P4HB, and liberated thioredoxin null P4HB. Protein lysates were harvested for immunoblot against main ER stress sensor and activator, BiP. Expression of ER-resident P4HB variants increase BiP expression significantly, whereas liberated versions are not significantly different than empty vector control. (FIG. 12A-12B) This suggests that the use of liberated P4HB can provide targeted clearance of TDP-43 pathology without activation of a global ER-stress response pathway which otherwise would further compound TDP-43-induced cell stress. The study provides validation that ER-P4HB overexpression and subsequent activation of the XBP1 transcriptional pathway leads to increase in ER-stress/UPR protein, BiP.

### Example 13: Nuclear TDP-43 Pathology Observed in Subset of ALS Cases

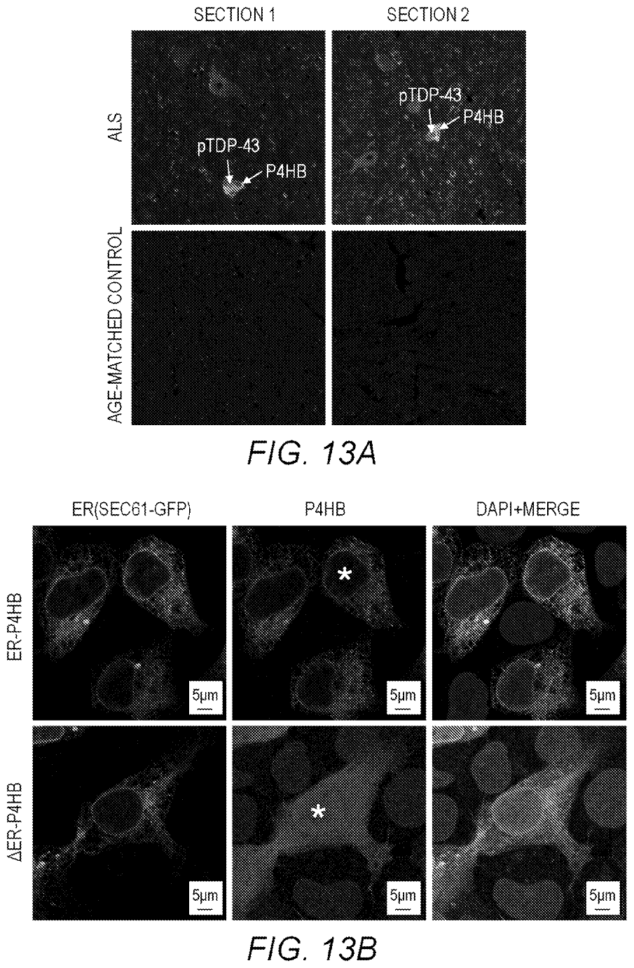

FIG. 13A shows immunohistochemistry of ALS-FTLD patient and age-matched control illustrating the presence of nuclear-localized TDP-43 pathology (pTDP-43) and anti-localized, ER-resident P4HB. ER-localization prevents nuclear entry of P4HB to site of nuclear TDP-43 pathology. FIG. 13B shows immunocytochemistry illustrating that by liberating P41-HB through mutation of ER-signal sequence, P4HB accesses the nucleus in addition to the cytoplasm (asterisk). Sec61 is used as an ER and nuclear lamin counterstain. The results of the study further support use of modified P4HB to increase accessibility to all possible sites of TDP-43 pathology.

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A modified protein disulfide isomerase (PDI) or a functional fragment thereof, wherein the modified PDI or functional fragment thereof comprises a deletion of an N-terminal endoplasmic reticulum (ER) signal sequence from a wild-type PDI or functional fragment thereof, wherein the PDI is not P4HB.

2. The modified PDI or functional fragment thereof of claim 1, wherein about 15 to about 50 amino acid residues are deleted from the N-terminus of the wild-type PDI or functional fragment thereof.

3. The modified PDI or functional fragment thereof of claim 1, wherein the deleted ER signal sequence from the N-terminus is replaced with an initiator methionine.

4. The modified PDI or functional fragment thereof of claim 1, wherein the deleted ER signal sequence from the N-terminus is replaced with an initiator methionine linked to an epitope tag.

5. A modified protein disulfide isomerase (PDI) or a functional fragment thereof, wherein the modified PDI or functional fragment thereof comprises a deletion of a C-terminal endoplasmic reticulum (ER) signal sequence from a wild-type PDI or functional fragment thereof.

6. A modified protein disulfide isomerase (PDI) or a functional fragment thereof, wherein the modified PDI or functional fragment thereof comprises a deletion of an endoplasmic reticulum (ER) signal sequence from a wild-type PDI or functional fragment thereof and further comprises a nuclear localization signal (NLS).

7. The modified PDI or functional fragment thereof of claim 1, wherein the modified PDI is a human PDI.

8. The modified PDI or functional fragment thereof of claim 1, wherein the modified PDI or functional fragment thereof is a PDI gene family member selected from the group of ERp29, PDIA2, and TXNDC12.

9. The modified PDI or functional fragment thereof of claim 5, wherein the modified PDI or functional fragment thereof is a PDI gene family member selected from the group of P4HB, ERp29, PDIA2, and TXNDC12.

10-13. (canceled)

14. A nucleic acid molecule encoding the modified PDI or functional fragment of claim 1.

15-18. (canceled)

19. A vector comprising the nucleic acid molecule of claim 14.

20-22. (canceled)

23. A host cell comprising the nucleic acid molecule of claim 14.

24-25. (canceled)

26. A pharmaceutical composition comprising the modified PDI or functional fragment thereof of claim 1 and a pharmaceutically acceptable carrier.

27. A method of treating a disease associated with TAR DNA-binding protein 43 (TDP-43) aggregation in a subject in need thereof, the method comprising administering to the subject a therapeutically effective amount of the modified protein disulfide isomerase (PDI) or a functional fragment thereof of claim 1, treating the disease.

28. (canceled)

29. A method of treating a disease associated with mutations in the TARDBP gene in a subject in need thereof, the method comprising administering to the subject a therapeutically effective amount of the modified protein disulfide isomerase (PDI) or a functional fragment thereof of claim 1 thereby treating the disease.

30-38. (canceled)

39. A method of delivering a modified PDI or functional fragment thereof to a subject, the method comprising administering to the subject the modified protein disulfide isomerase (PDI) or a functional fragment thereof of claim 1, thereby delivering the modified PDI or functional fragment thereof.

40-43. (canceled)

44. A method of disaggregating protein inclusions comprising TDP-43 in a subject, the method comprising administering to the subject the modified protein disulfide isomerase (PDI) or a functional fragment thereof of claim 1, thereby disaggregating protein inclusions comprising TDP-43.

45. (canceled)

46. A method of inhibiting formation of protein inclusions comprising TDP-43 in a subject, the method comprising administering to the subject the modified protein disulfide isomerase (PDI) or a functional fragment thereof of claim 1, thereby inhibiting formation of protein inclusions comprising TDP-43.

47. (canceled)

48. A method of restoring function of TDP-43 in a subject, the method comprising administering to the subject the modified protein disulfide isomerase (PDI) or a functional fragment thereof of claim 1, thereby restoring function of TDP-43.

49-50. (canceled)


SEQUENCE LISTING (27 sequences)

SEQ ID NO: 1 - protein, 175 aa, Homo sapiens
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 2 - protein, 166 aa, Homo sapiens
MMLHSALGLCLLLVTVSSNLAIAIKKEKRPPQTLSRGWGDDITWVQTYEEGLFYAQKSKKPLMVIHHLEDCQYSQALKKVFAQNEEIQEMAQNKFIMLNLMHETTDKNLSPDGQYVPRIMFVDPSLTVRADIAGRYSNRLYTYEPRDLPLLIENMKKALRLIQSEL

SEQ ID NO: 3 - protein, 172 aa, Homo sapiens
METRPRLGATCLLGFSFLLLVISSDGHNGLGKGFGDHIHWRTLEDGKKEAAASGLPLMVIIHKSWCGACKALKPKFAESTEISELSHNFVMVNLEDEEEPKDEDFSPDGGYIPRILFLDPSGKVHPEIINENGNPSYKYFYVSAEQVVQGMKEAQERLTGDAFRKKHLEDEL

SEQ ID NO: 4 - protein, 396 aa, Homo sapiens
MSATDRMGPRAVPGLRLALLLLLVLGTPKSGVQGQEGLDFPEYDGVDRVINVNAKNYKNVFKKYEVLALLYHEPPEDDKASQRQFEMEELILELAAQVLEDKGVGFGLVDSEKDAAVAKKLGLTEVDSMYVFKGDEVIEYDGEFSADTIVEFLLDVLEDPVELIEGERELQAFENIEDEIKLIGYFKSKDSEHYKAFEDAAEEFHPYIPFFATFDSKVAKKLTLKLNEIDFYEAFMEEPVTIPDKPNSEEEIVNFVEEHRRSTLRKLKPESMYETWEDDMDGIHIVAFAEEADPDGFEFLETLKAVAQDNTENPDLSIIWIDPDDFPLLVPYWEKTFDIDLSAPQIGVVNVTDADSVWMEMDDEEDLPSAEELEDWLEDVLEGEINTEDDDDDDDD

SEQ ID NO: 5 - protein, 399 aa, Homo sapiens
MKRTHLFIVGIYFLSSCRAEEGLNFPTYDGKDRVVSLSEKNFKQVLKKYDLLCLYYHEPVSSDKVTQKQFQLKEIVLELVAQVLEHKAIGFVMVDAKKEAKLAKKLGFDEEGSLYILKGDRTIEFDGEFAADVLVEFLLDLIEDPVEIISSKLEVQAFERIEDYIKLIGFFKSEDSEYYKAFEEAAEHFQPYIKFFATFDKGVAKKLSLKMNEVDFYEPFMDEPIAIPNKPYTEEELVEFVKEHQRPTLRRLRPEEMFETWEDDLNGIHIVAFAEKSDPDGYEFLEILKQVARDNTDNPDLSILWIDPDDFPLLVAYWEKTFKIDLFRPQIGVVNVTDADSVWMEIPDDDDLPTAEELEDWIEDVLSGKINTEDDDEDDDDDDNSDEEDNDDSDDDDDE

SEQ ID NO: 6 - protein, 747 aa, Homo sapiens
MGVWLNKDDYIRDLKRIILCFLIVYMAILVGTDQDFYSLLGVSKTASSREIRQAFKKLALKLHPDKNPNNPNAHGDFLKINRAYEVLKDEDLRKKYDKYGEKGLEDNQGGQYESWNYYRYDFGIYDDDPEIITLERREFDAAVNSGELWFVNFYSPGCSHCHDLAPTWRDFAKEVDGLLRIGAVNCGDDRMLCRMKGVNSYPSLFIFRSGMAPVKYHGDRSKESLVSFAMQHVRSTVTELWTGNFVNSIQTAFAAGIGWLITFCSKGGDCLTSQTRLRLSGMLFLNSLDAKEIYLEVIHNLPDFELLSANTLEDRLAHHRWLLFFHFGKNENSNDPELKKLKTLLKNDHIQVGRFDCSSAPDICSNLYVFQPSLAVFKGQGTKEYEIHHGKKILYDILAFAKESVNSHVTTLGPQNFPANDKEPWLVDFFAPWCPPCRALLPELRRASNLLYGQLKFGTLDCTVHEGLCNMYNIQAYPTTVVFNQSNIHEYEGHHSAEQILEFIEDLMNPSVVSLTPTTFNELVTQRKHNEVWMVDFYSPWCHPCQVLMPEWKRMARTLTGLINVGSIDCQQYHSFCAQENVQRYPEIRFFPPKSNKAYHYHSYNGWNRDAYSLRIWGLGFLPQVSTDLTPQTFSEKVLQGKNHWVIDFYAPWCGPCQNFAPEFELLARMIKGKVKAGKVDCQAYAQTCQKAGIRAYPTVKFYFYERAKRNFQEEQINTRDAKAIAALISEKLETLRNQGKRNKDEL

SEQ ID NO: 7 - protein, 273 aa, Homo sapiens
MEAAPSRFMFLLFLLTCELAAEVAAEVEKSSDGPGAAQEPTWLTDVPAAMEFIAATEVAVIGFFQDLEIPAVPILHSMVQKFPGVSFGISTDSEVLTHYNITGNTICLFRLVDNEQLNLEDEDIESIDATKLSRFIEINSLHMVTEYNPVTVIGLFNSVIQIHLLLIMNKASPEYEENMHRYQKAAKLFQGKILFILVDSGMKENGKVISFFKLKESQLPALAIYQTLDDEWDTLPTAEVSVEHVQNFCDGFLSGKLLKENRESEGKTPKVEL

SEQ ID NO: 8 - protein, 261 aa, Homo sapiens
MAAAVPRAAFLSPLLPLLLGFLLLSAPHGGSGLHTKGALPLDTVTFYKVIPKSKFVLVKFDTQYPYGEKQDEFKRLAENSASSDDLLVAEVGISDYGDKLNMELSEKYKLDKESYPVFYLFRDGDFENPVPYTGAVKVGAIQRWLKGQGVYLGMPGCLPVYDALAGEFIRASGVEARQALLKQGQDNLSSVKETQKKWAEQYLKIMGKILDQGEDFPASEMTRIARLIEKNKMSDGKKEELQKSLNILTAFQKKGAEKEEL

SEQ ID NO: 9 - protein, 406 aa, Homo sapiens
MHPAVFLSLPDLRCSLLLLVTWVFTPVTTEITSLDTENIDEILNNADVALVNFYADWCRFSQMLHPIFEEASDVIKEEFPNENQVVFARVDCDQHSDIAQRYRISKYPTLKLFRNGMMMKREYRGQRSVKALADYIRQQKSDPIQEIRDLAEITTLDRSKRNIIGYFEQKDSDNYRVFERVANILHDDCAFLSAFGDVSKPERYSGDNIIYKPPGHSAPDMVYLGAMTNFDVTYNWIQDKCVPLVREITFENGEELTEEGLPFLILFHMKEDTESLEIFQNEVARQLISEKGTINFLHADCDKFRHPLLHIQKTPADCPVIAIDSFRHMYVFGDFKDVLIPGKLKQFVFDLHSGKLHREFHHGPDPTDTAPGEQAQDVASSPPESSFQKLAPSEYRYTLLRDRDEL

SEQ ID NO: 10 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 11 - protein, 525 aa, Homo sapiens
MSRQLLPVLLLLLLRASCPWGQEQGARSPSEEPPEEEIPKEDGILVLSRHTLGLALREHPALLVEFYAPWCGHCQALAPEYSKAAAVLAAESMVVTLAKVDGPAQRELAEEFGVTEYPTLKFFRNGNRTHPEEYTGPRDAEGIAEWLRRRVGPSAMRLEDEAAAQALIGGRDLVVIGFFQDLQDEDVATFLALAQDALDMTFGLTDRPRLFQQFGLTKDTVVLFKKFDEGRADFPVDEELGLDLGDLSRFLVTHSMRLVTEFNSQTSAKIFAARILNHLLLFVNQTLAAHRELLAGFGEAAPRFRGQVLFVVVDVAADNEHVLQYFGLKAEAAPTLRLVNLETTKKYAPVDGGPVTAASITAFCHAVLNGQVKPYLLSQEIPPDWDQRPVKTLVGKNFEQVAFDETKNVFVKFYAPWCTHCKEMAPAWEALAEKYQDHEDIIIAELDATANELDAFAVHGFPTLKYFPAGPGRKVIEYKSTRDLETFSKFLDNGGVLPTEEPPEEPAAPFPEPPANSTMGSKEEL

SEQ ID NO: 12 - protein, 505 aa, Homo sapiens
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 13 - protein, 645 aa, Homo sapiens
MRPRKAFLLLLLLGLVQLLAVAGAEGPDEDSSNRENAIEDEEEEEEEDDDEEEDDLEVKEENGVLVLNDANFDNFVADKDTVLLEFYAPWCGHCKQFAPEYEKIANILKDKDPPIPVAKIDATSASVLASRFDVSGYPTIKILKKGQAVDYEGSRTQEEIVAKVREVSQPDWTPPPEVTLVLTKENFDEVVNDADIILVEFYAPWCGHCKKLAPEYEKAAKELSKRSPPIPLAKVDATAETDLAKRFDVSGYPTLKIFRKGRPYDYNGPREKYGIVDYMIEQSGPPSKEILTLKQVQEFLKDGDDVIIIGVFKGESDPAYQQYQDAANNLREDYKFHHTFSTEIAKFLKVSQGQLVVMQPEKFQSKYEPRSHMMDVQGSTQDSAIKDFVLKYALPLVGHRKVSNDAKRYTRRPLVVVYYSVDFSFDYRAATQFWRSKVLEVAKDFPEYTFAIADEEDYAGEVKDLGLSESGEDVNAAILDESGKKFAMEPEEFDSDTLREFVTAFKKGKLKPVIKSQPVPKNNKGPVKVVVGKTFDSIVMDPKKDVLIEFYAPWCGHCKQLEPVYNSLAKKYKGQKGLVIAKMDATANDVPSDRYKVEGFPTIYFAPSGDKKNPVKFEGGDRDLEHLSKFIEEHATKLSRTKEEL

SEQ ID NO: 14 - protein, 519 aa, Homo sapiens
MARAGPAWLLLAIWVVLPSWLSSAKVSSLIERISDPKDLKKLLRTRNNVLVLYSKSEVAAENHLRLLSTVAQAVKGQGTICWVDCGDAESRKLCKKMKVDLSPKDKKVELFHYQDGAFHTEYNRAVTFKSIVAFLKDPKGPPLWEEDPGAKDVVHLDSEKDFRRLLKKEEKPLLIMFYAPWCSMCKRMMPHFQKAATQLRGHAVLAGMNVYSSEFENIKEEYSVRGFPTICYFEKGRFLFQYDNYGSTAEDIVEWLKNPQPPQPQVPETPWADEGGSVYHLTDEDFDQFVKEHSSVLVMFHAPWCGHCKKMKPEFEKAAEALHGEADSSGVLAAVDATVNKALAERFHISEFPTLKYFKNGEKYAVPVLRTKKKFLEWMQNPEAPPPPEPTWEEQQTSVLHLVGDNFRETLKKKKHTLVMFYAPWCPHCKKVIPHFTATADAFKDDRKIACAAVDCVKDKNQDLCQQEAVKGYPTFHYYHYGKFAEKYDSDRTELGFTNYIRALREGDHERLGKKKEEL

SEQ ID NO: 15 - protein, 492 aa, Homo sapiens
MRRDLREKLVWVCRPLAPVEVPANISSDFQPCSPTSPAHSLSRKSPIMYPSTTMANAPGLVSCTFFLAVNGLYSSSDDVIELTPSNFNREVIQSDSLWLVEFYAPWCGHCQRLTPEWKKAATALKDVVKVGAVDADKHHSLGGQYGVQGFPTIKIFGSNKNRPEDYQGGRTGEAIVDAALSALRQLVKDRLGGRSGGYSSGKQGRSDSSSKKDVIELTDDSFDKNVLDSEDVWMVEFYAPWCGHCKNLEPEWAAAASEVKEQTKGKVKLAAVDATVNQVLASRYGIRGFPTIKIFQKGESPVDYDGGRTRSDIVSRALDLFSDNAPPPELLEIINEDIAKRTCEEHQLCVVAVLPHILDTGAAGRNSYLEVLLKLADKYKKKMWGWLWTEAGAQSELETALGIGGFGYPAMAAINARKMKFALLKGSFSEQGINEFLRELSFGRGSTAPVGGGAFPTIVEREPWDGRDGELPVEDDIDLSDVELDDLGKDEL

SEQ ID NO: 16 - protein, 584 aa, Homo sapiens
MDLLWMPLLLVAACVSAVHSSPEVNAGVSSIHITKPVHILEERSLLVLTPAGLTQMLNQTRFLMVLFHNPSSKQSRNLAEELGKAVEIMGKGKNGIGFGKVDITIEKELQQEFGITKAPELKLFFEGNRSEPISCKGVVESAALVVWLRRQISQKAFLFNSSEQVAEFVISRPLVIVGFFQDLEEEVAELFYDVIKDFPELTFGVITIGNVIGRFHVTLDSVLVFKKGKIVNRQKLINDSTNKQELNRVIKQHLTDFVIEYNTENKDLISELHIMSHMLLFVSKSSESYGIIIQHYKLASKEFQNKILFILVDADEPRNGRVFKYFRVTEVDIPSVQILNLSSDARYKMPSDDITYESLKKFGRSFLSKNATKHQSSEEIPKYWDQGLVKQLVGKNFNVVVFDKEKDVFVMFYAPWSKKCKMLFPLLEELGRKYQNHSTIIIAKIDVTANDIQLMYLDRYPFFRLFPSGSQQAVLYKGEHTLKGFSDFLESHIKTKIEDEDELLSVEQNEVIEEEVLAEEKEVPMMRKGLPEQQSPELENMTKYVSKLEEPAGKKKTSEEVVVVVAKPKGPPVQKKKPKVKEEL

SEQ ID NO: 17 - protein, 280 aa, Homo sapiens
MAPSGSLAVPLAVLVLLLWGAPWTHGRRSNVRVITDENWRELLEGDWMIEFYAPWCPACQNLQPEWESFAEWGEDLEVNIAKVDVTEQPGLSGRFIITALPTIYHCKDGEFRRYQGPRTKKDFINFISDKEWKSIEPVSSWFGPGSVLMSSMSALFQLSMWIRTCHNYFIEDLGLPVWGSYTVFALATLFSGLLLGLCMIFVADCLCPSKRRRPQPYPYPSKKLLSESAQPLKKVEEEQEADEEDVSEEEAESKEGTNKDFPQNAIRQRSLGPSLATDKS

SEQ ID NO: 18 - protein, 258 aa, Homo sapiens
MAVLAPLIALVYSVPRLSRWLAQPYYLLSALLSAAFLLVRKLPPLCHGLPTQREDGNPCDFDWREVEILMFLSAIVMMKNRRSMFLMTCKPPLYMGPEYIKYFNDKTIDEELERDKRVTWIVEFFANWSNDCQSFAPIYADLSLKYNCTGLNFGKVDVGRYTDVSTRYKVSTSPLTKQLPTLILFQGGKEAMRRPQIDKKGRAVSWTFSEENVIREFNLNELYQRAKKLSKAGDNIPEEQPVASTPTTVSDGENKKDK

SEQ ID NO: 19 - protein, 195 aa, Homo sapiens
MAAWKSWTALRLCATVVVLDMVVCKGFVEDLDESFKENRNDDIWLVDFYAPWCGHCKKLEPIWNEVGLEMKSIGSPVKVGKMDATSYSSIASEFGVRGYPTIKLLKGDLAYNYRGPRTKDDIIEFAHRVSGALIRPLPSQQMFEHMQKRHRVFFVYVGGESPLKEKYIDAASELIVYTYFFSASEEVVPEVIFKI

SEQ ID NO: 20 - protein, 349 aa, Homo sapiens
MAGGRCGPQLTALLAAWIAAVAATAGPEEAALPPEQSRVQPMTASNWTLVMEGEWMLKFYAPWCPSCQQTDSEWEAFAKNGEILQISVGKVDVIQEPGLSGRFFVTTLPAFFHAKDGIFRRYRGPGIFEDLQNYILEKKWQSVEPLTGWKSPASLTMSGMAGLFSISGKIWHLHNYFTVTLGIPAWCSYVFFVIATLVFGLFMGLVLVVISECFYVPLPRHLSERSEQNRRSEEAHRAEQLQDAEEEKDDSNEEENKDSLVDDEEEKEDLGDEDEAEEEEEEDNLAAGVDEERSEANDQGPPGEDGVTREEVEPEEAEEGISEQPCPADTEVVEDSLRQRKSQHADKGL

SEQ ID NO: 21 - protein, 324 aa, Homo sapiens
MEDAKVYVAKVDCTAHSDVCSAQGVRGYPTLKLFKPGQEAVKYQGPRDFQTLENWMLQTLNEEPVTPEPEVEPPSAPELKQGLYELSASNFELHVAQGDHFIKFFAPWCGHCKALAPTWEQLALGLEHSETVKIGKVDCTQHYELCSGNQVRGYPTLLWFRDGKKVDQYKGKRDLESLREYVESQLQRTETGATETVTPSEAPVLAAEPEADKGTVLALTENNFDDTIAEGITFIKFYAPWCGHCKTLAPTWEELSKKEFPGLAGVKIAEVDCTAERNICSKYSVRGYPTLLLFRGGKKVSEHSGGRDLDSLHRFVLSQAKDEL

SEQ ID NO: 22 - protein, 4 aa, synthetic construct
KDEL

SEQ ID NO: 23 - protein, 4 aa, synthetic construct
RDEL

SEQ ID NO: 24 - protein, 4 aa, synthetic construct
HDEL

SEQ ID NO: 25 - protein, 4 aa, synthetic construct
KEEL

SEQ ID NO: 26 - protein, 4 aa, synthetic construct
KVEL

SEQ ID NO: 27 - protein, 17 aa, synthetic construct
KRKMDETDASSAVKVKR